(19) United States
(12) Patent Application Publication
(10) Pub. No.: US 2021/0290452 A1
(43) Pub. Date: Sep. 23, 2021

(54) SYSTEMS AND METHODS FOR TREATING AN ARTICLE INCLUDING A BIODEGRADABLE SUPERABSORBENT MATERIAL

(71) Applicant: Tethis, Inc., Raleigh, NC (US)

(72) Inventors: Ryan Nicholas Chan, Raleigh, NC (US); Gordon Sidney Cox, Durham, NC (US); Hailey Katherine Davis, Raleigh, NC (US); Rachel Laura Preston, Raleigh, NC (US); Robin Weitkamp, Raleigh, NC (US); Kimberly Weitkamp, Raleigh, NC (US)

(73) Assignee: Tethis, Inc., Raleigh, NC (US)

(21) Appl. No.: 17/204,521

(22) Filed: Mar. 17, 2021

Related U.S. Application Data
(60) Provisional application No. 62/992,432, filed on Mar. 20, 2020.

(51) Int. Cl. A61F 13/53; A61L 15/32; A61L 15/38; A61L 15/60

(52) U.S. Cl. CPC A61F 13/53; A61L 15/32; A61L 15/38; A61L 2300/254; A61F 2013/530795; A61L 2300/604; A61L 15/60

(57) ABSTRACT

A system for treating used hygienic articles including soiled or swollen superabsorbent materials (SAMs). The portion of the article including the soiled or swollen SAM is treated with a chemical which restructures or degrades the soiled or swollen SAM so that the products of the reaction are easily disposable in a method alternative to trash disposal and landfilling, including through municipal treatment where the products of the reaction do not clog a standard toilet or standard plumbing. Hygienic articles include baby diapers, adult diapers, incontinence pads, menstrual pads, or any other article which includes one or more SAMs.

### CROSS-REFERENCES TO RELATED APPLICATIONS

This application is related to and claims priority from the following applications. This application claims priority to and the benefit of U.S. Provisional App. No. 62/992,432, filed Mar. 20, 2021, which is hereby incorporated herein by reference in its entirety.

### BACKGROUND OF THE INVENTION

1. Field of the Invention

The present invention relates to absorbent hygienic articles, and more specifically to systems and methods for treating used hygienic articles including superabsorbent materials such that the hygienic article or a portion of the hygienic article is flushable in a standard toilet and does not clog standard plumbing waste lines, with the treated hygienic article or treated portion of the hygienic article which is flushed being treatable by existing standard municipal waste water treatment facilities.

2. Description of the Prior Art

It is generally known in the prior art to provide for disposal of and recycling of hygienic articles including superabsorbent materials.

U.S. Pat. No. 6,569,137 for Absorbent incontinence pads by inventor Suzuki, filed Jan. 12, 2000 and issued May 27, 2003, is directed to an absorbent incontinence pad with a liquid impervious air permeable back sheet and an absorbent unit partly covered by the back sheet, wherein the absorbent unit has a non-woven fabric substrate, an absorbent zone formed by a plurality of highly absorbent layers extending in the form of bands on the surface of the non-woven fabric substrate and an air permeable zone where no such highly absorbent layer exists, which has sufficiently adequate properties to meet incontinence requirements and provides a comfortable feeling during use.

U.S. Pat. No. 10,538,878 for Method for recovering pulp fibers from used absorbent article by inventor Konishi, filed Jun. 9, 2017 and issued Jan. 21, 2020, is directed to a method for recovering pulp fibers having little damage from a used absorbent article which includes a water permeable front sheet, a water impermeable back sheet and an absorbent body that contains pulp fibers and a superabsorbent polymer. At least one opening with a circle equivalent diameter of 5-45 mm, or 10-45 mm cut, is made in the front sheet and/or the back sheet of the used absorbent article, which is then agitated in an organic acid aqueous solution with a pH of less than or equal to 2.5, and the superabsorbent polymer is deactivated and the pulp fibers and superabsorbent polymer are discharged from the used absorbent article through the opening or cut.

US Patent Publication No. 20180272395 for A Method of Processing Waste Material Including A Super Absorbent Polymer by inventor Herriott, filed Oct. 3, 2016 and published Sep. 27, 2018, is directed to a method of processing waste material including a super absorbent polymer including: shredding the waste material; applying a salt to the shredded waste material to deactivate the super absorbent polymer; dewatering the waste material following deactivation of the super absorbent polymer; applying a liquid biocide to the shredded waste material. The preferred salt is aluminum sulphate. The step of applying a liquid biocide to the shredded waste is provided after the step of dewatering the waste material in the preferred form.

U.S. Pat. No. 7,838,610 for Ion-sensitive super-absorbent polymer by inventor Adachi, filed Sep. 22, 2006 and issued November 23, 2010, is directed to a super-absorbent resin which can be used to design an absorbing core or absorbing goods capable of being flown into a flushing toilet. The present invention relates to a super-absorbent resin having as a main component thereof a repeating unit having an ionic dissociation group in its main or side chain, wherein said resin has absorption capacity without load to saline solution (CRCs) for 4 hours of not smaller than 10 g/g, and solubility in ion-exchanged water of not lower than 50% by weight.

US Patent Publication No. 20040126585 for Water dispersible commode/bedpan liner by inventor Kerins, filed Dec. 27, 2002 and published Jul. 1, 2004, is directed to a water dispersible commode/bedpan liner. The liner is a film useful as a flushable commode or bedpan liner. The liner may be positioned before use and then placed in a toilet afterwards where it is flushed. The film is a two layer co-extruded film. At least half of the film is a predominately water soluble polymer. No more than half of the film is a skin fluid barrier layer of an extrudable polymer that may be biodegradable. The film also includes a chemical that is activated by tap water to help cause the film to break apart.

U.S. Pat. No. 5,415,643 for Flushable absorbent composites by inventor Kolb, filed Dec. 7, 1992 and issued May 16, 1995, is directed to absorbent composite structures containing fluff pulp and a superabsorbent material, such as those useful for disposable diapers, can be made to be flushable if the superabsorbent has the requisite properties. It has been found that flushability of such composite structures is enhanced when using superabsorbent materials having a ratio of the Absorbency Under Load (AUL) to the Centrifuge Retention Capacity (CRC) of about 0.70 or greater.

U.S. Pat. No. 5,952,251 for Coformed dispersible nonwoven fabric bonded with a hybrid system by inventor Jackson, filed Jun. 30, 1995 and issued Sep. 14, 1999, is directed to a water-dispersible coformed fibrous nonwoven fabric structure comprising a primary reinforcing polymer material, preferably capable of being meltspun; a secondary reinforcing polymer material having an average fiber length less than or equal to about 15 mm and preferably having a softening point at least about 30° C. lower than the softening point of the primary reinforcing polymer; and, an absorbent material, such as pulp or a superabsorbent. The fabric structure maintains desired tensile strength and softness while being water-dispersible and flushable. The fabric produced can be incorporated into an article and can be flushed down a commode. The fabric is flushable when placed in water, with agitation, if necessary, and will disperse into unrecognizable pieces without clogging conventional plumbing or piping. A method of producing the fabric structure comprises mixing the secondary reinforcing material and absorbent material and injecting this coform blend into a stream of meltspun primary reinforcing fibers. After a web structure has been established, the structure is exposed to thermal or ultrasonic energy sufficient to soften and bond the secondary reinforcing material fibers, but not to soften the primary reinforcing material fibers. An embossed pattern can be printed on the structure.

U.S. Pat. No. 8,436,058 for Methods for separation and conditioning of products containing super absorbent polymers by inventor Grimes, filed May 16, 2009 and issued May 7, 2013, is directed to a method for separating a product comprising a super absorbent polymer, a fiber and a plastic to separate the product into components thereof, the method comprising adding water to the product, and pressing the product in order to separate the product into components comprising a plastics component and a super absorbent polymer and fiber component. Other methods of the present disclosure include a method for producing a reusable plastic, reusable paper fiber stream and a reusable super absorbent polymer from the treatment of a product comprising a super absorbent polymer, a fiber and a plastic. Still other embodiments include a method for the treatment of wet super absorbent polymer, comprising salt assisted dehydration.

U.S. Pat. No. 8,821,687 for Flushable article including polyurethane binder and method of using the same by inventor Muvundamina, filed Dec. 9, 2011 and issued Sep. 2, 2014, is directed to a flushable article such as a wipe that includes a substrate that includes fibers, and a dried binder composition in contact with the fibers, the article (e.g., wipe) being insoluble in water having a pH of no greater than 6, and disintegrating in water having a pH of at least 6.5.

U.S. Pat. No. 5,300,358 for Degradable absorbant structures by inventor Evers, filed Nov. 24, 1992 and issued Apr. 5, 1994, is directed to compostable and flushable absorbent structures for sanitary uses for the absorption of body fluids comprising an absorbent degradable fibrous core and a backsheet that is cold-water soluble but water impermeable.

US Patent Publication No. 20060154054 for A flushable body fluid absorbent composite by inventor Banks, filed Jan. 13, 2005 and published Jul. 13, 2006, is directed to a flushable bodily liquid absorbent composite product, having a bodily liquid absorbent core and a backing layer applied to a garment side of the core. The backing layer is readily soluble in cold water and has a water impervious layer on its core side and a water resistant layer on its garment facing side.

### SUMMARY OF THE INVENTION

The present invention relates to systems and methods for treating used hygienic articles including superabsorbent materials.

In one embodiment, the present invention is directed to a system for degrading a biodegradable superabsorbent material including an absorbent material including the biodegradable superabsorbent material, and a degradation agent, wherein the degradation agent is operable to permanently deactivate the particles of the biodegradable superabsorbent material such that the particles of the deactivated biodegradable superabsorbent material are not operable to swell or reswell under free swell conditions.

In another embodiment, the present invention is directed to a system for degrading a biodegradable superabsorbent material including an absorbent material including the biodegradable superabsorbent material and a degradation agent, wherein the degradation agent is activated when the absorbent material is exposed to water, and wherein the degradation agent is operable to permanently deactivate the particles of the biodegradable superabsorbent material such that the particles of the deactivated biodegradable superabsorbent material are not operable to swell or reswell under free swell conditions.

In yet another embodiment, the present invention is directed to a system for degrading a biodegradable superabsorbent material including the biodegradable superabsorbent material and a degradation agent, wherein the degradation agent is operable to permanently deactivate the particles of the biodegradable superabsorbent material such that the particles of the deactivated biodegradable superabsorbent material are not operable to swell or reswell under free swell conditions.

These and other aspects of the present invention will become apparent to those skilled in the art after a reading of the following description of the preferred embodiment when considered with the drawings, as they support the claimed invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a baby diaper shell according to one embodiment of the present invention.

FIG. 2 illustrates a removable, disposable insert operable to be inserted into the baby diaper shell of FIG. 1 according to one embodiment of the present invention.

FIG. 3 illustrates a menstrual pad including a removable, absorbent core with biocompostable properties according to one embodiment of the present invention.

FIG. 4 illustrates an incontinence product including a removable, absorbent core with biocompostable properties according to one embodiment of the present invention.

FIG. 5 is a photograph of the residue of a test sample of a swollen SAM treated with a restructuring agent after being filtered and the residue of a control sample of a swollen SAM not treated with a restructuring agent after being filtered.

FIG. 6 is a photograph of the residues of the samples of FIG. 5 after soaking in water for approximately 18 hours and being filtered a second time.

FIG. 7 is a photograph of the residue of a test sample of a swollen SAM treated with a restructuring agent after being filtered and the residue of a control sample of a swollen SAM not treated with a restructuring agent after being filtered.

FIG. 8 is a photograph of the residues of the samples of FIG. 7 after soaking in water for approximately 18 hours and being filtered a second time.

FIG. 9 is a photograph of the residue of a test sample of a swollen SAM treated with a restructuring agent after being filtered and the residue of a control sample of a swollen SAM not treated with a restructuring agent after being filtered.

FIG. 10 is a photograph of the residues of the samples of FIG. 9 after soaking in water for approximately 18 hours and being filtered a second time.

FIG. 11 is a photograph of the residue of a test sample of a swollen SAM treated with a restructuring agent after being filtered and the residue of a control sample of a swollen SAM not treated with a restructuring agent after being filtered.

FIG. 12 is a photograph of the residues of the samples of FIG. 11 after soaking in water for approximately 18 hours and being filtered a second time.

FIG. 13 is a photograph of the residue of a test sample of a swollen SAM treated with a restructuring agent after being filtered and the residue of a control sample of a swollen SAM not treated with a restructuring agent after being filtered.

FIG. 14 is a photograph of the residues of the samples of FIG. 13 after soaking in water for approximately 18 hours and being filtered a second time.

FIG. 15 is a photograph of the residue of a test sample of a swollen SAM treated with a restructuring agent after being filtered and the residue of a control sample of a swollen SAM not treated with a restructuring agent after being filtered.

FIG. 16 is a photograph of the residues of the samples of FIG. 15 after soaking in water for approximately 18 hours and being filtered a second time.

FIG. 17 is a photograph of the residue of a test sample of a swollen SAM treated with a restructuring agent after being filtered and the residue of a control sample of a swollen SAM not treated with a restructuring agent after being filtered.

FIG. 18 is a photograph of the residues of the samples of FIG. 17 after soaking in water for approximately 18 hours and being filtered a second time.

FIG. 19 is a photograph of the residue of a test sample of a swollen SAM treated with a restructuring agent after being filtered and the residue of a control sample of a swollen SAM not treated with a restructuring agent after being filtered.

FIG. 20 is a photograph of the residues of the samples of FIG. 19 after soaking in water for approximately 18 hours and being filtered a second time.

FIG. 21 is a photograph of a test sample of a swollen SAM treated with a restructuring agent and the residue of a control sample of a swollen SAM not treated with a restructuring agent after being filtered.

FIG. 22 is a photograph of the residues of the samples of FIG. 21 after soaking in water for approximately 18 hours and being filtered.

FIG. 23 is a photograph of the residues of samples of swollen SAMs treated with a restructuring agent, filtered, sitting in water for approximately 18 hours, and being filtered a second time.

### DETAILED DESCRIPTION

The present invention is generally directed to systems and methods for treating used hygienic articles including soiled or swollen superabsorbent materials (SAMs), such as soiled or swollen superabsorbent polymers (SAPs). The invention generally includes a chemical including an enzyme, a gel breaker, an oxidizing agent, or the like for treating the used hygienic article or a portion of the used hygienic article and parameters under which the used hygienic article is treated including duration and temperature. The treatment provides for changing the structure of the soiled or swollen SAM in the hygienic article so that the treated, used hygienic article or a treated portion of the used hygienic article is flushable in a standard toilet without causing blockage of the toilet or plumbing, including plumbing waste lines and sewer pipes, with the flushed treated hygienic article or flushed treated portion of the hygienic article being further treatable by existing standard municipal waste water treatment facilities to be degraded into bioavailable materials.

In one embodiment, the present invention is directed to a system for degrading a biodegradable superabsorbent material including an absorbent material including the biodegradable superabsorbent material, and a degradation agent, wherein the degradation agent is operable to permanently deactivate the particles of the biodegradable superabsorbent material such that the particles of the deactivated biodegradable superabsorbent material are not operable to swell or reswell under free swell conditions.

In another embodiment, the present invention is directed to a system for degrading a biodegradable superabsorbent material including an absorbent material including the biodegradable superabsorbent material and a degradation agent, wherein the degradation agent is activated when the absorbent material is exposed to water, and wherein the degradation agent is operable to permanently deactivate the particles of the biodegradable superabsorbent material such that the particles of the deactivated biodegradable superabsorbent material are not operable to swell or reswell under free swell conditions.

In yet another embodiment, the present invention is directed to a system for degrading a biodegradable superabsorbent material including the biodegradable superabsorbent material and a degradation agent, wherein the degradation agent is operable to permanently deactivate the particles of the biodegradable superabsorbent material such that the particles of the deactivated biodegradable superabsorbent material are not operable to swell or reswell under free swell conditions.

None of the prior art discloses a system or method for treating used hygienic articles including soiled or swollen SAMs to change the structure of the soiled or swollen SAM such that the used hygienic article or a portion of the used hygienic article does not clog the toilet or plumbing when flushed, and wherein and the treated hygienic article or treated portion of the used hygienic article is treatable by existing standard municipal waste water treatment facilities.

Currently, consumers use a wide variety of hygienic articles including diapers, incontinence pads, and menstrual pads. These articles are typically manufactured out of material which does not biodegrade and ends up in landfills. In fact, according to the Environmental Protection Agency, approximately 20 billion disposable diapers are added to landfills in the United States each year, which amounts to about 3.5 million tons of waste. Due to the large amount of waste created through the use of diapers, there has been a recent trend towards using reusable diapers when possible. However, reusable diapers generally have several disadvantages, including being time consuming to clean and increased energy consumption associated with cleaning diapers as opposed to simply disposing of the diapers in the trash.

While recent advancements have focused on increasing the biodegradability or compostability of diapers, many components of the diapers are still not biodegradable or compostable. Additionally, diapers with biodegradable elements are still typically thrown in the trash or a separate receptable where the diapers are not chemically treated. This results in unpleasant odors in the house as well as increased household waste or the need for a service to pick up the used diapers.

Prior attempts at making diapers or components of diapers flushable have not been successful. Diapers, or the portions of diapers which collect waste, typically contain SAMs to aid in the retention of fluids. Swollen or soiled SAMs tend to swell and clump together when placed in the toilet and often clog plumbing. Prior art solutions to making hygienic articles or portions of hygienic articles flushable have generally focused on using lower amounts of SAMs in the hygienic articles to avoid issues with clogging plumbing. This approach creates ineffective hygienic articles which often leak or otherwise exhibit inferior performance to articles with an adequate amount of SAM for absorbing bodily fluids. For instance, certain commercially available diapers currently include flushable cores with approximately 3.2 grams of SAM out of a total core weight of approximately 29.5 grams. Accordingly, these cores include approximately 10.8% SAM by weight. Typical non-flushable absorbent cores weigh approximately 20 grams, with 55% to 60% of the weight of the core being SAM.

While many diaper recycling processes involve deactivating or otherwise chemically treating SAMs included in diapers, this deactivation or other chemical treatment typically occurs under extreme conditions such as extreme temperature or extreme pressure. Accordingly, chemically treating used diapers in this manner to deactivate or otherwise chemically alter SAMs is not cost efficient or even possible for the majority of consumers of diapers. Other recycling processes include applying a salt to articles including SAMs, often after the articles have been otherwise mechanically and/or chemically treated. Although application of a salt typically reduces the absorption capacity and/or retention capacity of a SAM, exposing the SAM to high salinity does not permanently deactivate the SAM. A SAM that is not permanently deactivated resumes swelling when exposed to a solution with a lower salinity, and the temporarily deactivated SAM is therefore not suitable for disposal in a standard toilet or in standard plumbing because of the high likelihood of swelling of the temporarily deactivated SAM. The temporarily deactivated SAM is also not suitable for treatment at a standard municipal waste water treatment plant because of the high likelihood of swelling of the temporarily deactivated SAM. Additionally, most untreated SAMs, including temporarily deactivated SAMs, swell at a pH between 5 and 9; the pH of municipal tap water and waste water fall within this range.

Accordingly, there is a long felt, unmet need for a system and method for treating hygienic articles such as diapers or portions of hygienic articles such as diapers so that these treated articles or treated portions of these articles are flushable in a standard toilet connected to standard plumbing and/or a standalone unit connected to standard plumbing such that the treated articles or treated portions of articles do not clog the toilet, the standalone unit, or the standard plumbing. Additionally, there is a long-felt unmet need for treating articles or portions of articles such that the treated articles or portions of articles are treatable by existing standard municipal waste water treatment facilities Specifically, the present invention provides for treating soiled or swollen SAMs in hygienic articles or portions of hygienic articles using chemicals such as oxidizing agents, gel breakers, bacteria, and enzymes to degrade the soiled or swollen SAMs or “restructure” the soiled or swollen SAMs such that the SAM which is treated with the chemicals or bacteria does not have the capacity to reabsorb liquid and has a sufficiently low percentage of particle sizes and sufficiently low viscosity such that the resulting product is flushable in a standard toilet without clogging the toilet or plumbing and the flushed product is treatable by existing standard municipal waste water treatment facilities.

Additionally, by degrading the SAM such that the SAM is not capable of swelling or reswelling in standard free swell conditions, the present invention provides for quicker degradation of the remainder of the materials in a biodegradable or biocompostable article. Typically, the degradation of absorbent articles such as cores which incorporate SAMs is slowed or initially prevented by the presence of SAMs which have not yet degraded. By intentionally degrading SAMs such that they cannot swell or reswell, the process of degradation of the remainder of the absorbent article is sped up considerably compared to absorbent articles in which the SAM is not degraded.

Referring now to the drawings in general, the illustrations are for the purpose of describing one or more preferred embodiments of the invention and are not intended to limit the invention thereto.

The present invention includes a system for treating used hygienic articles including a used hygienic article which incorporates a soiled or swollen SAM and a chemical which degrades the soiled or swollen SAM, or restructures the soiled or swollen SAM such that the resulting product of the treatment has a permanently reduced absorption capacity and permanently reduced retention capacity compared to the unswollen and unsoiled SAM or does not have any absorption capacity or retention capacity. Hygienic articles include baby diapers, adult diapers, incontinence pads, menstrual pads, or any other article which includes one or more SAMs.

In one embodiment, the present invention includes a restructuring chemical or degradation agent as part of the core of a hygienic article. The restructuring chemical is operable to be blended with another chemical such as one or more SAMs in the core or applied as a separate layer in the core. In another embodiment, the restructuring chemical is not included in the core or in any part of the hygienic article. The restructuring chemical is operable to be provided in a variety of forms, including powder, liquid, gel, gel pack, tabs, capsules, or any other form known in the art. Additionally, the restructuring chemical is operable to be packaged using a variety of different packaging, such as blister packs. The restructuring chemical is provided with the hygienic article in one embodiment. Alternatively, the restructuring chemical is provided separately from the hygienic article.

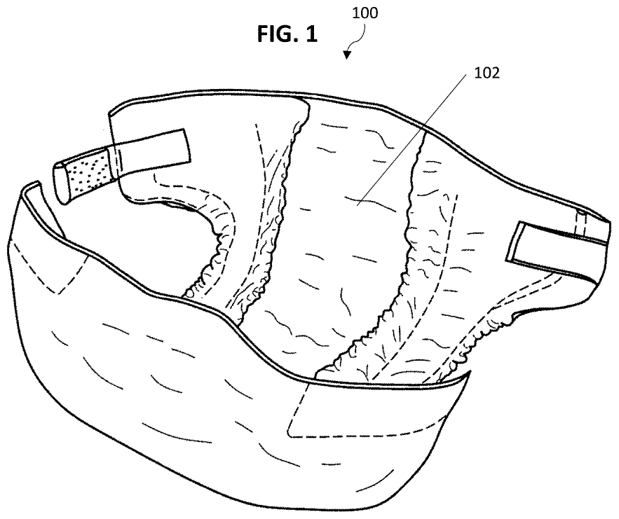

FIG. 1 illustrates a baby diaper shell 100 according to one embodiment of the present invention. The diaper shell 100 includes a fluid impermeable liner sheet 102 to prevent leakage from a removable, absorbent core which is operable to be inserted into the diaper shell 100 on top of the fluid impermeable liner sheet 102. The baby diaper shell 100, including the fluid impermeable liner sheet 102, is operable to be biodegradable or non-biodegradable. Alternatively, the baby diaper shell 100, including the impermeable liner sheet 102, is biodegradable and disposable via flushing in a standard toilet after treatment with a restructuring agent. The baby diaper shell 100, including the impermeable liner sheet 102, is also treatable by existing standard municipal waste water treatment facilities.

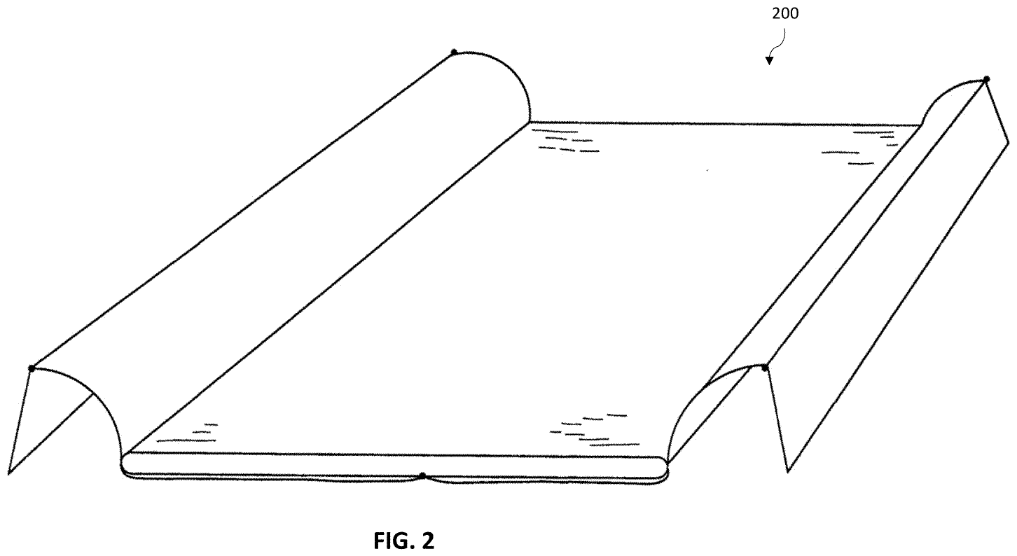

FIG. 2 illustrates a removable, absorbent core 200 for a baby diaper shell according to one embodiment of the present invention. The removable, absorbent core 200 includes a SAM. Preferably, the SAM is blended with a restructuring agent which causes restructuring of the SAM or degradation of the SAM when the restructuring agent is activated such that the treated SAM has a lower viscosity and/or a smaller average particle size than the untreated SAM. In one example, the viscosity of a solution including the biodegradable superabsorbent material and the degradation agent decreases by at least 85% over 5 minutes. In an exemplary embodiment, the SAM and restructuring agent blend includes approximately 1% to 2% restructuring agent and approximately 99% to 98% SAM. Other ratios of restructuring agents and SAMs are also envisioned according to the present disclosure, such as less than approximately 5% restructuring agent and greater than approximately 95% SAM. In yet another embodiment, a ratio of 25% restructuring agent to 75% SAM is utilized. In this embodiment, the SAM is fully degraded within 2 minutes of exposure to the restructuring agent such that the treated SAM is not operable to swell or reswell in free swell conditions. The restructuring agent is activated when the removable, absorbent core 200 including the restructuring agent is exposed to water, such as when the removable, absorbent core 200 is placed in the water of a toilet bowl or water in another receptacle. The removable absorbent core 200 is preferably constructed of biodegradable material. Examples of biodegradable absorbent materials include cellulose fluff, wood fluff, or cotton. As used throughout the present specification, the term “absorbent material” refers to a material or layer included in an article which is capable of absorbing fluid. This term is used in contrast to the term “superabsorbent material”, with the term “superabsorbent material” referring to a material or layer which includes a superabsorbent polymer or other material which is classified as a superabsorbent. Alternatively, the removable, absorbent core 200 is constructed from “fluffless” or alternative non-woven materials such as a web of airlaid fabric with natural materials. In yet another embodiment, the removable, absorbent core 200 is constructed of a synthetic fluff or synthetic fluffless material. Examples of synthetic fluff material include polyester, polyethylene, or polypropylene. In one embodiment, the airlaid fabric is similar to the materials disclosed in U.S. Pat. No. 5,445,777 by inventor Noel, et al., which is incorporated herein by reference in its entirety. In one embodiment, the core matrix is at least approximately 50% airlaid fabric. In another embodiment, the core matrix is at least approximately 65% airlaid fabric. In a further embodiment, the core matrix is at least approximately 85% airlaid fabric. In yet another embodiment, the core matrix is between 50% and 100% airlaid fabric, wherein between 0% and 50% of the core matrix includes an adhesive, a bonding agent, and/or a SAM. Bonding agents in one embodiment include resins, latex emulsions, and/or thermoplastic fibers. In another embodiment, bonding agents include one or more binders which are insoluble at a pH less than 6.5 but are soluble at a pH greater than 6.5. Examples of these binders are described in U.S. Pat. No. 8,821,687 by inventor Muvundamina, which is incorporated herein by reference in its entirety. The removable, absorbent core is preferably removable from the rest of the diaper such that the removable, absorbent core is operable to be chemically treated via a restructuring agent and/or mechanically treated via a restructuring agent and then flushed in a standard toilet such that the treated component of the diaper does not clog standard plumbing (e.g. plumbing with a 3 inch diameter or greater) or disposed of in another manner. If flushed, the removable absorbent core is preferably treatable by existing standard municipal waste water treatment facilities.

Examples of removable cores or other removable components of diapers are described in US Patent Publication No. 20100179497 by inventor Brownlee, US Patent Publication No. 20180338871 by inventor Richardson, U.S. Pat. No. 5,415,643 by inventor Kolb, U.S. Pat. No. 8,449,518 by inventor Allison-Rogers, and U.S. Pat. No. 8,002,762 by inventor Allison-Rogers, each of which is incorporated herein by reference in its entirety. In one embodiment, the removable, absorbent core includes a biodegradable top layer or is wrapped in a biodegradable layer to prevent direct contact of the skin of the baby of the diaper with the removable, absorbent core 200. In one embodiment, the absorbent core includes a top layer of a non-woven hydrophilic material or is wrapped in the non-woven hydrophilic material. In one embodiment, the removable, absorbent core 200 includes a semi-permeable outer liner constructed of cellulose. More specifically, the outer liner is constructed of regenerated cellulose such as viscose rayon in one embodiment.

In another embodiment, the core includes a SAM to fluff ratio of approximately 60 to approximately 40. The core density is preferably between approximately 0.18 to 0.2, and weighs approximately 21 grams. Alternatively, the core weighs 24 grams or 27 grams with a SAM to fluff ratio of approximately 60 to approximately 40 and a core density between approximately 0.18 to 0.2.

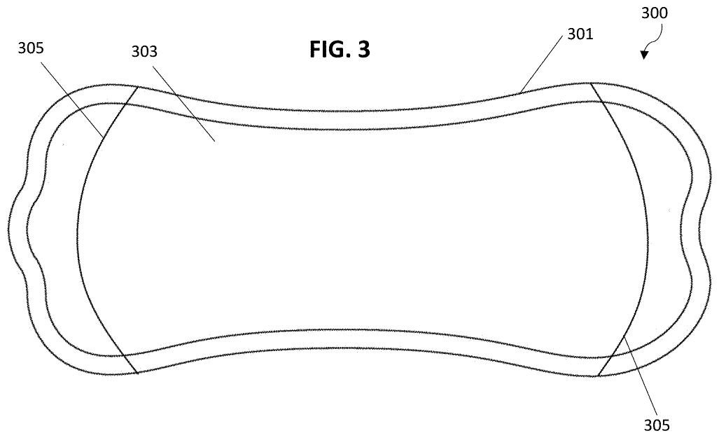

FIG. 3 illustrates a menstrual pad including a removable, absorbent core with biocompostable properties according to one embodiment of the present invention. The menstrual pad 300 includes a liquid impermeable backsheet 301, a removable, absorbent core 303, and elastic bands 305 to keep the removable, absorbent core 303 in place. The removable, absorbent core 303 is constructed of any material recited with respect to the removable, absorbent core of the baby diaper depicted in FIG. 2. The removable, absorbent core 303 preferably includes one or more biodegradable and/or synthetic SAMs as described herein. In one embodiment, a rear of the hygienic pad 300 includes an adhesive for attachment to a clothing article. Preferably, the adhesive is biodegradable, compostable, and/or recyclable, such as a soy or starch-based adhesive. In one embodiment, the menstrual pad 300 is reusable and the removable, absorbent core 303 is removed, treated with a restructuring agent, and disposed through flushing in a standard toilet. The treated removable, absorbent core 303 is also treatable by existing standard municipal waste water treatment facilities. Alternatively, the entire menstrual pad 300 is biodegradable and disposable via flushing in a standard toilet after treatment with a restructuring agent. The entire menstrual pad 300 is also treatable by existing standard municipal waste water treatment facilities. In one embodiment, the removable, absorbent core includes a biodegradable top layer or is wrapped in a biodegradable layer to prevent direct contact of the skin of the user with the removable, absorbent core 303. In one embodiment, the absorbent core includes a top layer of a non-woven hydrophilic material or is wrapped in the non-woven hydrophilic material. In one embodiment, the removable, absorbent core 303 includes a semi-permeable outer liner constructed of cellulose. More specifically, the outer liner is constructed of regenerated cellulose such as viscose rayon in one embodiment. Alternatively, an absorbent pad or an absorbent core includes one or more SAMs on a first side and an adhesive for attachment to an undergarment on a second side, and is constructed out of any of the materials described herein. Preferably, the adhesive is biodegradable, compostable, and/or recyclable, such as a soy or starch-based adhesive.

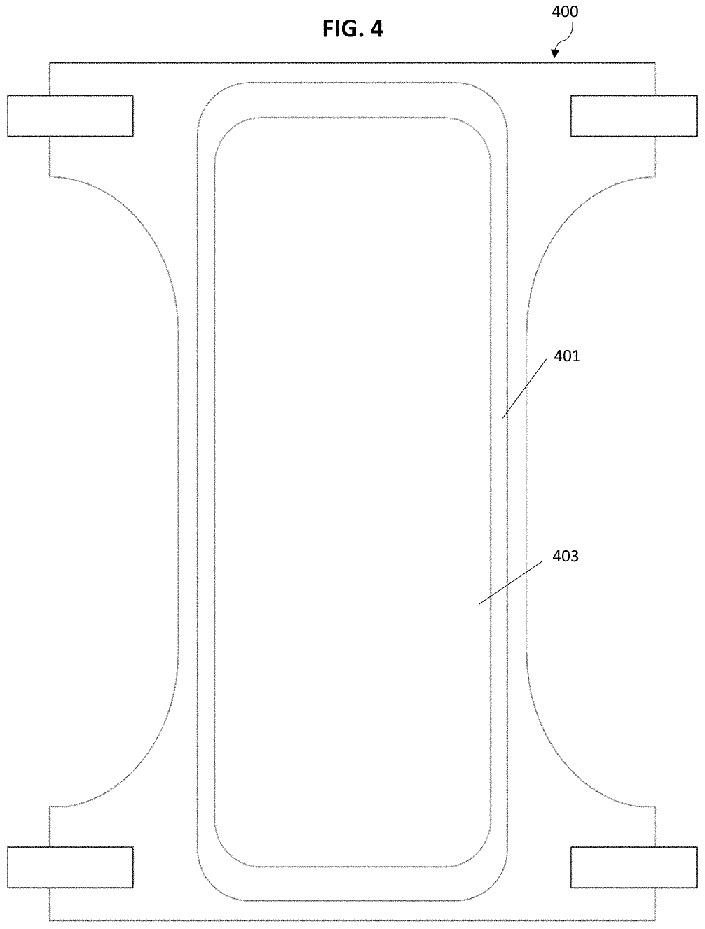

FIG. 4 illustrates an incontinence product including a removable, absorbent core with biocompostable properties according to one embodiment of the present invention. The incontinence product 400 includes a fluid impermeable liner sheet 401 operable to receive a removable, absorbent core 403. The removable, absorbent core 403 is constructed of any material recited with respect to the removable, absorbent core of the baby diaper depicted in FIG. 2. The removable, absorbent core 403 preferably includes one or more biodegradable and/or synthetic SAMs as described herein. In one embodiment, a rear of the incontinence product 400 includes an adhesive for attachment to a clothing article. Preferably, the adhesive is biodegradable, compostable, and/or recyclable, such as a soy or starch-based adhesive. In one embodiment, the incontinence product 400 is reusable and the removable, absorbent core 403 is removed, treated with a restructuring agent, and disposed through flushing in a standard toilet. The treated removable, absorbent core 403 is also treatable by existing standard municipal waste water treatment facilities. Alternatively, the incontinence product 400 is biodegradable and disposable via flushing in a standard toilet after treatment with a restructuring agent. The entire incontinence product 400 is also treatable by existing standard municipal waste water treatment facilities. In one embodiment, the removable, absorbent core 403 includes a biodegradable top layer or is wrapped in a biodegradable layer such as a non-woven hydrophilic material layer to prevent direct contact of the skin of the user of the incontinence product 400 with the removable, absorbent core 403. In one embodiment, the removable, absorbent core 403 includes a semi-permeable outer liner constructed of cellulose. More specifically, the outer liner is constructed of regenerated cellulose such as viscose rayon in one embodiment.

Preferably, the SAM utilized in the removable, absorbent core is a biodegradable SAM. In a preferred embodiment, the biodegradable SAM includes one or more biodegradable SAPs described in US Patent Publication No. 2020/0054782, US Patent Publication No. 2021/0045942, and US Patent Publication No. 2021/0022931 by inventor Chan, each of which is incorporated herein by reference in its entirety. Examples of these biodegradable SAPs include modified cross-linked starch-based biopolymers. The biodegradable SAP preferably has a Free Swell Capacity (F SC) of at least 25 g/g, a Centrifuge Retention Capacity (CRC) of at least 16 g/g, and an Absorption Against Pressure (AAP) of at least 6 g/g in a saline solution. Alternatively, the biodegradable SAP has a FSC, CRC, and AAP or AUL of any SAP described in US Patent Publication No. 2020/0054782, US Patent Publication No. 2021/0045942, and US Patent Publication No. 2021/0022931. In another embodiment, the biodegradable SAP includes any biodegradable SAP known in the art. Known biodegradable SAP include, but are not limited to, biopolymers based on starches (including amylose and/or amylopectin), chitosans, hemicelluloses, lignins, celluloses, chitins, alginates, dextrans, pullanes, polyhydroxyalkanoates, fibrins, cyclodextrins, proteins (e.g., soy protein), other polysaccharides (e.g., pectin), and/or polylactic acids. The biodegradable SAP is also operable to be present as part of a blend of biodegradable SAP/non-biodegradable SAP described in US Patent Publication No. 2020/0054782, US Patent Publication No. 2021/0045942, and US Patent Publication No. 2021/0022931. Alternatively, the SAM is operable to be a synthetic SAM, a non-biodegradable SAM, or a blend of synthetic SAMs or non-biodegradable SAMs.

Notably, the present invention provides for treatment of soiled or used removable absorbents such as absorbent cores including soiled or swollen synthetic SAMs, soiled or swollen biodegradable SAMs, and combinations thereof. The present invention preferably provides for reduction of the absorption capacity and retention capacity of soiled or swollen SAMs through a variety of mechanisms. In some embodiments, the present invention utilizes reactions which decrease the pH such that a salt is converted to an acid, cause chemical degradation by breaking ether bonds in glucose or breaking ester crosslinks, attach divalent or trivalent metals to carboxylic acids of SAMs, and/or other reactions which change the chemical structure of soiled or swollen SAMs. These reactions utilize enzymes, oxidizing agents, gel breakers, and/or other chemicals which restructure the soiled or swollen SAMs such that the soiled or swollen SAMs have significantly reduced absorption capacity and reduced retention capacity or do not have any absorption capacity or retention capacity. In the present specification, “restructure” or “degrade” means to alter the physical or chemical structure of the soiled or swollen SAM such that the soiled or swollen SAM has a reduced absorption capacity or a reduced retention capacity compared to the untreated SAM under free swell conditions. Under the present invention, restructuring is accomplished via exposure to chemicals which cause physical and/or chemical changes to the soiled or swollen SAMs. Chemicals which perform restructuring or degradation are referred to as restructuring agents, oxidizing agents, gel breakers, and degradation agents throughout the present application. The change to the chemical structure or the physical change to the SAM is preferably irreversible. In some embodiments, the chemical structure of the soiled or swollen SAMs is changed by breaking the ether bonds between glucose units in the soiled or swollen SAM or the ester crosslinks in the soiled or swollen SAM.

Different chemicals are utilized to treat different soiled or swollen SAMs to reduce the swelling properties of the soiled or swollen SAMs according to the present invention. In one embodiment, biodegradable modified starch-based biopolymers including those described in US Patent Publication No. 2020/0054782 are treated with an amylase solution or a potassium peroxymonosulfate solution to lower the viscosity of the biodegradable modified starch-based biopolymer. Specifically, treating a biodegradable modified starch-based biopolymer with amylase, such as in Examples 1-3 below, lowers the viscosity of a solution including the swollen SAM or degrades a gel including the swollen SAM by breaking ether bonds between glucose units in amylose and/or amylopectin of the swollen SAM. In other words, treatment of the biodegradable modified starch-based biopolymer with amylase causes the treated biodegradable modified starch-based biopolymer to have a reduced absorption capacity or a reduced retention capacity compared to the untreated biodegradable modified starch-based biopolymer. Treating a biodegradable modified starch-based biopolymer with potassium peroxymonosulfate, sodium hypochlorite, or hydrogen peroxide, such as in Examples 5-7 below lowers the viscosity of a solution including the swollen SAM or degrades a gel including the swollen SAM by breaking ether bonds between glucose units in amylose and/or amylopectin or breaking the swollen SAM ester crosslinks. In other words, treatment of the biodegradable modified starch-based biopolymer with potassium peroxymonosulfate, sodium hypochlorite, or hydrogen peroxide causes the treated biodegradable modified starch-based biopolymer to have a reduced absorption capacity or a reduced retention capacity compared to the untreated biodegradable modified starch-based biopolymer. Treating a biodegradable modified starch-based biopolymer with magnesium sulfate as described in Example 4 causes divalent magnesium to be attached to the carboxylic acid groups of the biodegradable modified starch-based biopolymer. The attachment of divalent magnesium to the carboxylic acid groups of the biodegradable modified starch-based biopolymer lowers the viscosity of a solution including the swollen SAM or degrades a gel including the swollen SAM. In other words, attachment of divalent magnesium to the carboxylic acid groups of the biodegradable modified starch-based biopolymer causes the treated biodegradable modified starch-based biopolymer to have a reduced absorption capacity or a reduced retention capacity compared to the untreated biodegradable modified starch-based biopolymer. In addition, hypochlorous acid in combination with sodium hydroxide is utilized to permanently reduce the absorption capacity or the retention capacity of swollen SAMs. Examples of restructuring agents are included in Table 1 below. The state of the restructuring agents when introduced to the SAM is indicated as either S (solid) or L (liquid).

TABLE 1
Ingredients of Restructuring Agents

| Restructuring | | | |
|---|---|---|---|
| Agent | Ingredients | Type | State |
| Non-chlorine | sodium percarbonate, sodium | Oxidizing | S |
| bleach | carbonate, and others | agent | |
| Cellulase | cellulase | Enzyme | S |
| Concentrated | sodium hypochlorite (8.25%), | Oxidizing | L |
| household bleach | Sodium chloride, sodium | agent | |
| | carbonate, sodium hydroxide | | |
| Enzymatic | sodium carbonate, sodium | Oxidizing | S |
| Detergent | percarbonate, lipase, protease, | agent and | |
| | mannanase | enzyme | |
| Septic Treatment | cellulase, lipase, protease, | Enzyme | L |
| with Enzymes | amylase | | |
| Borax Powder | sodium tetraborate | Oxidizing | S |
| | | agent | |
| Amylase | amylase | Enzymes | S |
| Septic System | wheat bran carrier, sodium | Bacteria & | S |
| Powder | chloride, bacillus lichenformis, | enzymes | |
| | bacillus subtilis, alpha amylase, | | |
| | protease | | |
| Spa Cleaner | 31% potassium | Oxidizing | S |
| | peroxymonosulfate | agent | |

Notably, the present invention does not require complete depolymerization of amylose and/or amylopectin to lower the viscosity of a solution including the swollen SAM or degrade a gel including the swollen SAM and to reduce the reswelling or swelling capacity of the SAM. Rather, partial depolymerization of amylose and/or amylopectin is operable to cause sufficient reduced free swell capacity of the SAM in some embodiments.

The following examples mimic swelling of the SAM when exposed to urine and treatment of the swollen SAM with a restructuring agent as well as any reswelling of the treated, swollen SAM that would occur when placing the treated, swollen SAM a toilet bowl filled with water. Notably, the following examples were all conducted at standard temperature and pressure. The reactions described below are operable to be accelerated through raising the temperature under which the reactions occur. However, the temperature is not raised to more than 140 degrees Fahrenheit. Additionally, catalysts are operable to be included in these reactions to expedite the reactions.

### EXAMPLE 1: MODIFIED STARCH-BASED BIOPOLYMER TREATED WITH LIQUID AMYLASE

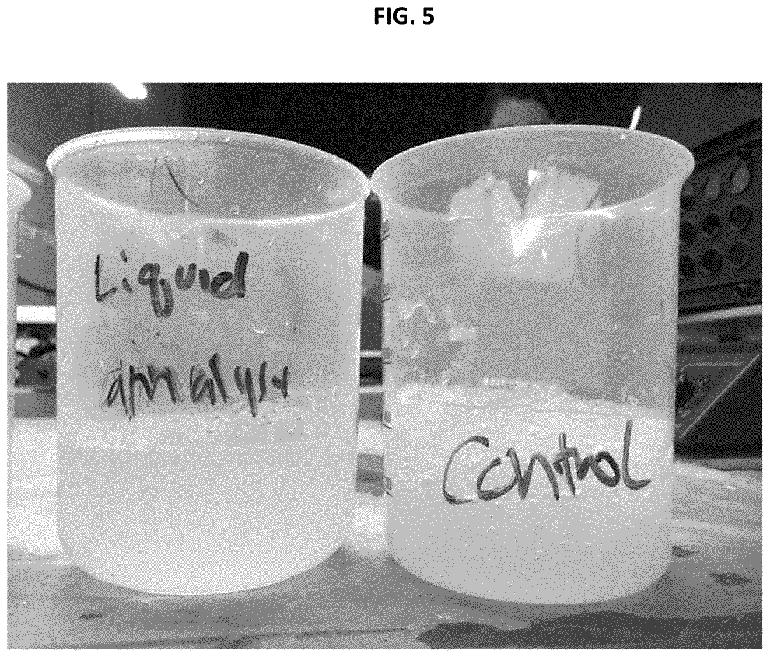

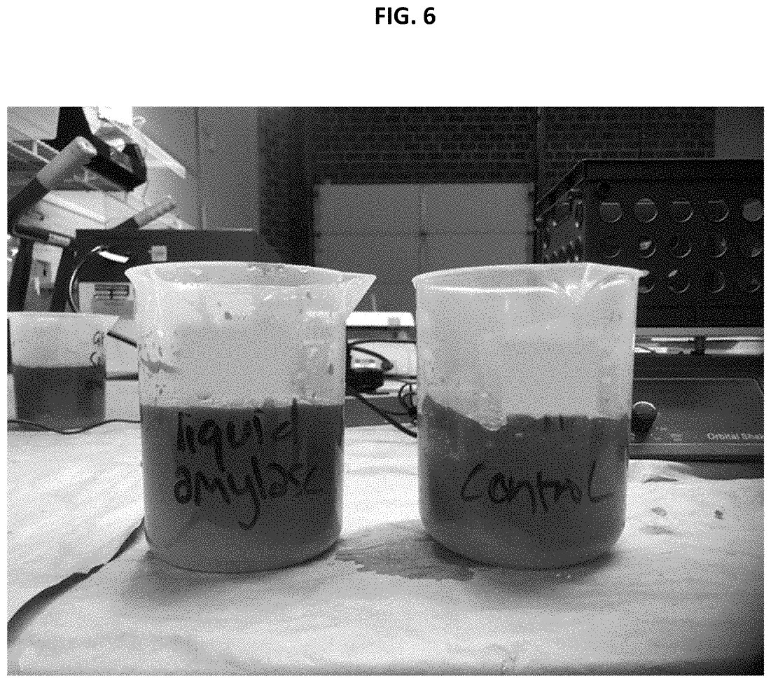

A test sample was prepared by swelling 15.0 grams of a modified starch-based biopolymer having a centrifuge retention capacity (CRC) of at least about 12 g/g or an absorbency under load (AUL) or Absorption Against Pressure (AAP) at 0.7 psi of at least about 8 g/g in a saline solution and an amylose content of less than about 85% w/w to about 90% using a 0.9 wt. % saline solution to mimic swelling of the SAM in an absorbent core of a diaper. Approximately 300 milliliters of water was added and 15.0 milliliters of liquid amylase was added such that the amylase was present in approximately a 1:1 weight ratio with the unswelled SAM. A control sample was also prepared by swelling 15.0 grams of the modified starch-based biopolymer utilized above to about 90% using a 0.9 wt. % saline solution to mimic swelling of the SAM in an absorbent core of a diaper. Approximately 300 milliliters of water was added to the control sample. The test sample and the control sample were stirred and visually analyzed for 30 minutes, then left to sit for approximately 6 hours. After sitting for approximately 6 hours, the samples were each filtered through 150-micron mesh sieves. FIG. 5 is a photograph of the residue of the samples after the samples were filtered through the 150-micron mesh sieves. The test sample is to the left of the control sample in FIG. 5. The samples were then transferred to approximately 2000 milliliters of water and were allowed to sit overnight, or for approximately 18 hours to provide time for any reswelling to occur. The samples were then filtered again through 150-micron mesh sieves. FIG. 6 is a photograph of the residue of the samples after the samples were allowed to sit for approximately 18 hours and filtered the second time through the 150-micron mesh sieves. The test sample is to the left of the control sample in FIG. 6. As is evident from FIG. 6, the residue of the test sample which was treated with liquid amylase was much less viscous than the control sample after soaking in water for approximately 18 hours. All prior steps were all performed at room temperature under standard pressure.

### EXAMPLE 2: MODIFIED STARCH-BASED BIOPOLYMER TREATED WITH SOLID AMYLASE

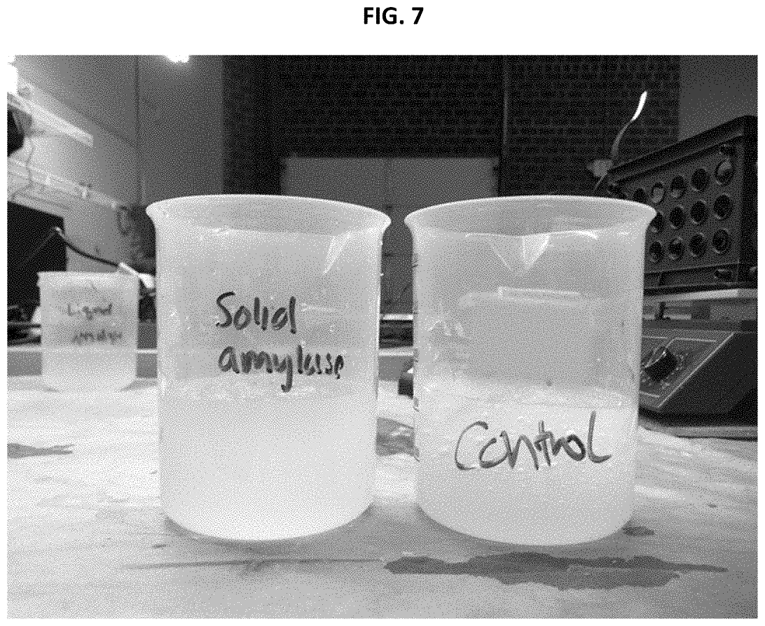

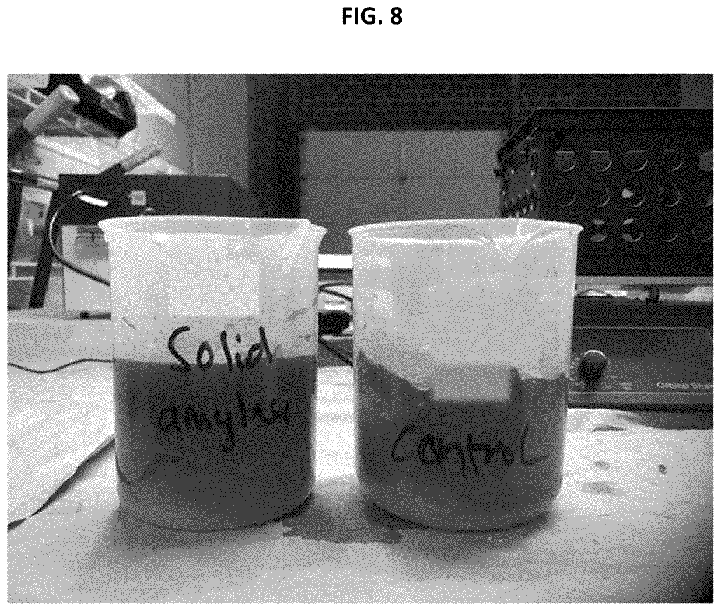

A test sample was prepared by swelling 15.0 grams of a modified starch-based biopolymer having a centrifuge retention capacity (CRC) of at least about 12 g/g or an absorbency under load (AUL) at 0.7 psi of at least about 8 g/g in a saline solution and an amylose content of less than about 85% w/w to about 90% using a 0.9 wt. % saline solution to mimic swelling of the SAM in an absorbent core of a diaper. Approximately 300 milliliters of water was added and 15.0 grams of solid amylase was added such that the amylase was present in approximately a 1:1 weight ratio with the unswelled SAM. A control sample was also prepared by swelling 15.0 grams of the modified starch-based biopolymer utilized above to about 90% using a 0.9 wt. % saline solution to mimic swelling of the SAM in an absorbent core of a diaper. Approximately 300 milliliters of water was added to the control sample. The test sample and the control sample were stirred and visually analyzed for 30 minutes, then left to sit for approximately 6 hours. After sitting for approximately 6 hours, the samples were each filtered through 150-micron mesh sieves FIG. 7 is a photograph of the residue of the samples after the samples were filtered through the 150-micron mesh sieves. The test sample is to the left of the control sample in FIG. 7. The samples were then transferred to approximately 2000 milliliters of water and were allowed to sit overnight, or for approximately 18 hours to provide time for any reswelling to occur. The samples were then filtered again through 150-micron mesh sieves. FIG. 8 is a photograph of the residue of the samples after the samples were allowed to sit for approximately 18 hours and filtered the second time through the 150-micron mesh sieves. The test sample is to the left of the control sample in FIG. 8. As is evident from FIG. 8, the residue of the test sample which was treated with solid amylase was much less viscous than the control sample after soaking in water for approximately 18 hours. All prior steps were all performed at room temperature under standard pressure.

### EXAMPLE 3: MODIFIED STARCH-BASED BIOPOLYMER TREATED WITH PROBIOTIC

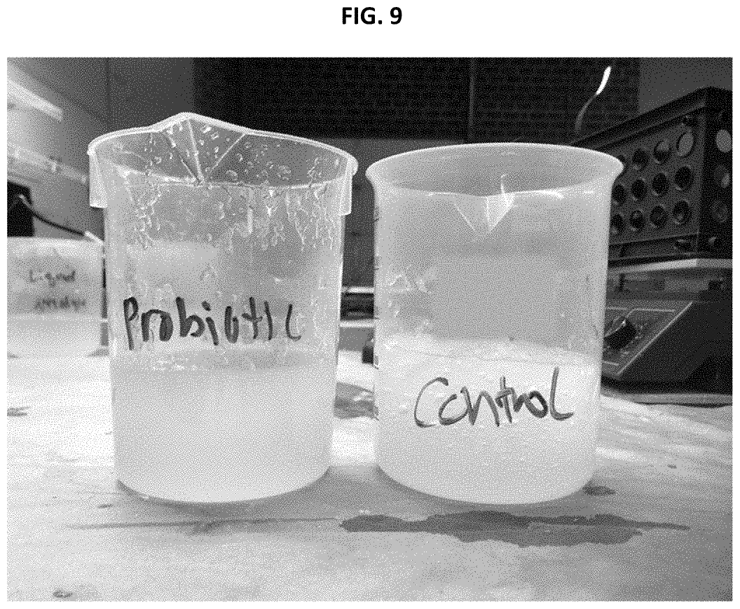

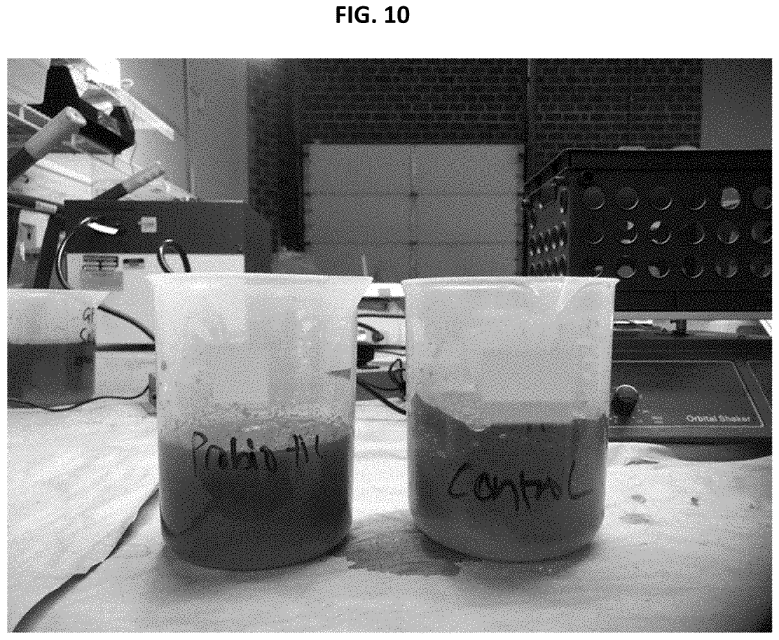

A test sample was prepared by swelling 15.0 grams of a modified starch-based biopolymer having a centrifuge retention capacity (CRC) of at least about 12 g/g or an absorbency under load (AUL) at 0.7 psi of at least about 8 g/g in a saline solution and an amylose content of less than about 85% w/w to about 90% using a 0.9 wt. % saline solution to mimic swelling of the SAM in an absorbent core of a diaper. Approximately 300 milliliters of water was added and 15 capsules of probiotic including 75,000 total dextrinizing units (DU) of amylase, 360,000 Hemoglobin Unit Tyrosine (HUT) of protease, 18,750 Federation Internationale Pharmaceutique units (FIP) of lipase, and 7,500 cellulase units (CU) of cellulase were broken open and added. A control sample was also prepared by swelling 15.0 grams of the modified starch-based biopolymer utilized above to about 90% using a 0.9 wt. % saline solution to mimic swelling of the SAM in an absorbent core of a diaper. Approximately 300 milliliters of water was added to the control sample. The test sample and the control sample were stirred and visually analyzed for 30 minutes, then left to sit for approximately 6 hours. After sitting for approximately 6 hours, the samples were each filtered through 150-micron mesh sieves. FIG. 9 is a photograph of the residue of the samples after the samples were filtered through the 150-micron mesh sieves. The test sample is to the left of the control sample in FIG. 9. The samples were then transferred to approximately 2000 milliliters of water and were allowed to sit overnight, or for approximately 18 hours to provide time for any reswelling to occur. The samples were then filtered again through 150-micron mesh sieves. FIG. 10 is a photograph of the residue of the samples after the samples were allowed to sit for approximately 18 hours and filtered the second time through the 150-micron mesh sieves. The test sample is to the left of the control sample in FIG. 10. As is evident from FIG. 10, the residue of the test sample which was treated with 15 capsules of probiotic including 75,000 DU of amylase, 360,000 HUT of protease, 18,750 FIP of lipase, and 7,500 CU of cellulase was much less viscous than the control sample after soaking in water for approximately 18 hours. All prior steps were all performed at room temperature under standard pressure.

### EXAMPLE 4: MODIFIED STARCH-BASED BIOPOLYMER TREATED WITH MAGNESIUM SULFATE

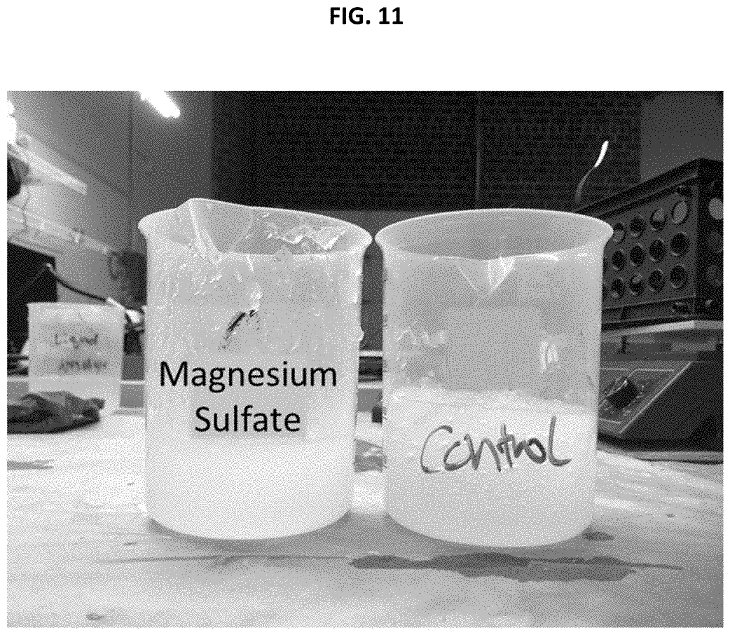

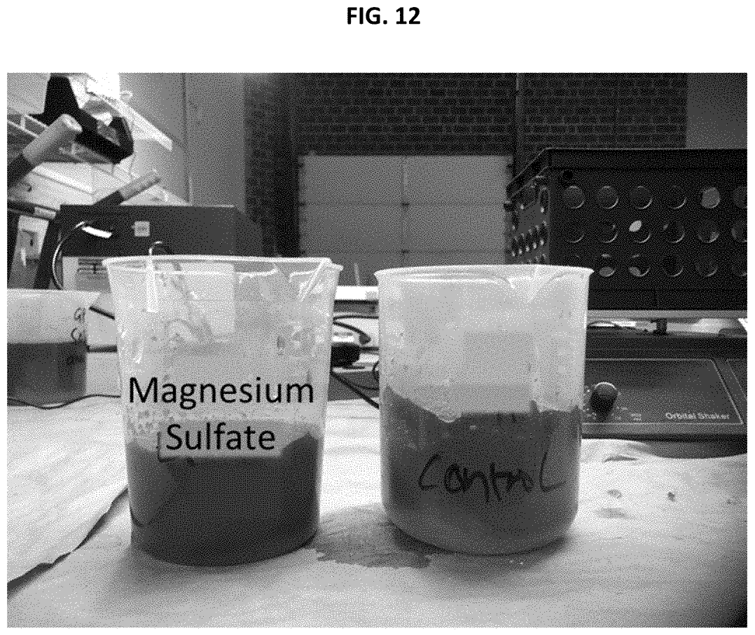

A test sample was prepared by swelling 15.0 grams of a modified starch-based biopolymer having a centrifuge retention capacity (CRC) of at least about 12 g/g or an absorbency under load (AUL) at 0.7 psi of at least about 8 g/g in a saline solution and an amylose content of less than about 85% w/w to about 90% using a 0.9 wt. % saline solution to mimic swelling of the SAM in an absorbent core of a diaper. Approximately 300 milliliters of water was added and 15.0 grams of magnesium sulfate (Epsom salt) was added such that the magnesium sulfate was present in approximately a 1:1 weight ratio with the unswelled SAM. A control sample was also prepared by swelling 15.0 grams of the modified starch-based biopolymer utilized above to about 90% using a 0.9 wt. % saline solution to mimic swelling of the SAM in an absorbent core of a diaper. Approximately 300 milliliters of water was added to the control sample. The test sample and the control sample were stirred and visually analyzed for 30 minutes, then left to sit for approximately 6 hours. After sitting for approximately 6 hours, the samples were each filtered through 150-micron mesh sieves. FIG. 11 is a photograph of the residue of the samples after the samples were filtered through the 150-micron mesh sieves. The test sample is to the left of the control sample in FIG. 11. The samples were then transferred to approximately 2000 milliliters of water and were allowed to sit overnight, or for approximately 18 hours to provide time for any reswelling to occur. The samples were then filtered again through 150-micron mesh sieves. FIG. 12 is a photograph of the residue of the samples after the samples were allowed to sit for approximately 18 hours and filtered the second time through the 150-micron mesh sieves. The test sample is to the left of the control sample in FIG. 12. As is evident from FIG. 12, the residue of the test sample which was treated with magnesium sulfate was much less viscous than the control sample after soaking in water for approximately 18 hours. However, some swelling was observed in the test sample which was treated with magnesium sulfate. All prior steps were all performed at room temperature under standard pressure.

### EXAMPLE 5: MODIFIED STARCH-BASED BIOPOLYMER TREATED WITH POTASSIUM PEROXYMONOSULFATE

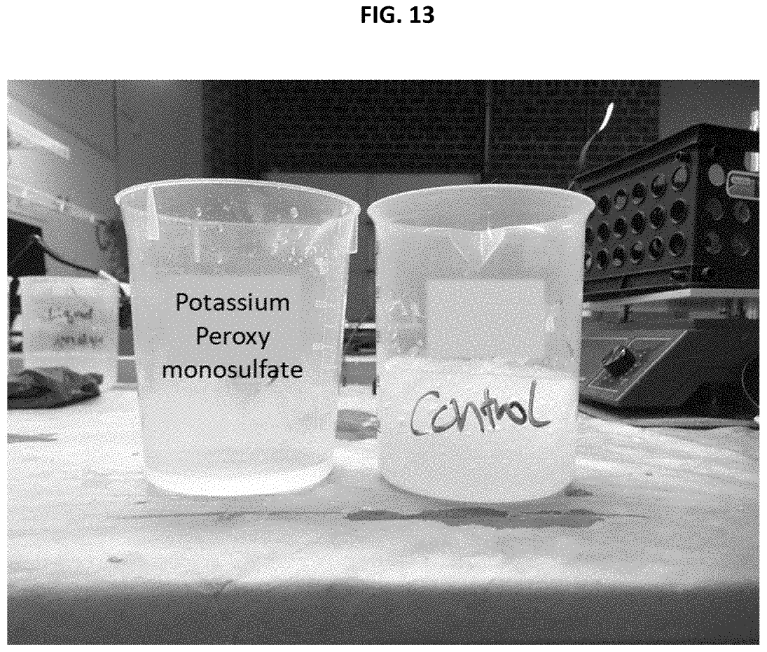

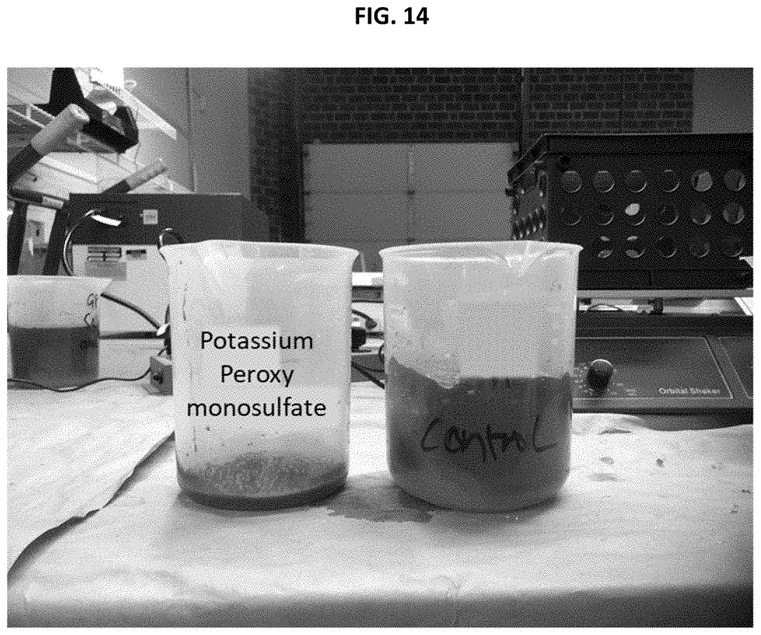

A test sample was prepared by swelling 15.0 grams of a modified starch-based biopolymer having a centrifuge retention capacity (CRC) of at least about 12 g/g or an absorbency under load (AUL) at 0.7 psi of at least about 8 g/g in a saline solution and an amylose content of less than about 85% w/w to about 90% using a 0.9 wt. % saline solution to mimic swelling of the SAM in an absorbent core of a diaper. Approximately 300 milliliters of water was added and 15.0 grams of a multipurpose spa cleaner comprising 31% potassium peroxymonosulfate w/w was added. A control sample was also prepared by swelling 15.0 grams of the modified starch-based biopolymer utilized above to about 90% using a 0.9 wt. % saline solution to mimic swelling of the SAM in an absorbent core of a diaper. Approximately 300 milliliters of water was added to the control sample. The test sample and the control sample were stirred and visually analyzed for 30 minutes, then left to sit for approximately 6 hours. After sitting for approximately 6 hours, the samples were each filtered through 150-micron mesh sieves. FIG. 13 is a photograph of the residue of the samples after the samples were filtered through the 150-micron mesh sieves. The test sample is to the left of the control sample in FIG. 13. The samples were then transferred to approximately 2000 milliliters of water and were allowed to sit overnight, or for approximately 18 hours to provide time for any reswelling to occur. The samples were then filtered again through 150-micron mesh sieves. FIG. 14 is a photograph of the residue of the samples after the samples were allowed to sit for approximately 18 hours and filtered the second time through the 150-micron mesh sieves. The test sample is to the left of the control sample in FIG. 14. As is evident from FIG. 14, the residue of the test sample which was treated with the multipurpose spa cleaner comprising 31% potassium peroxymonosulfate w/w was much less viscous than the control sample after soaking in water for approximately 18 hours. All prior steps were all performed at room temperature under standard pressure.

### EXAMPLE 6: MODIFIED STARCH-BASED BIOPOLYMER TREATED WITH HYDROGEN PEROXIDE (3 WT. %)

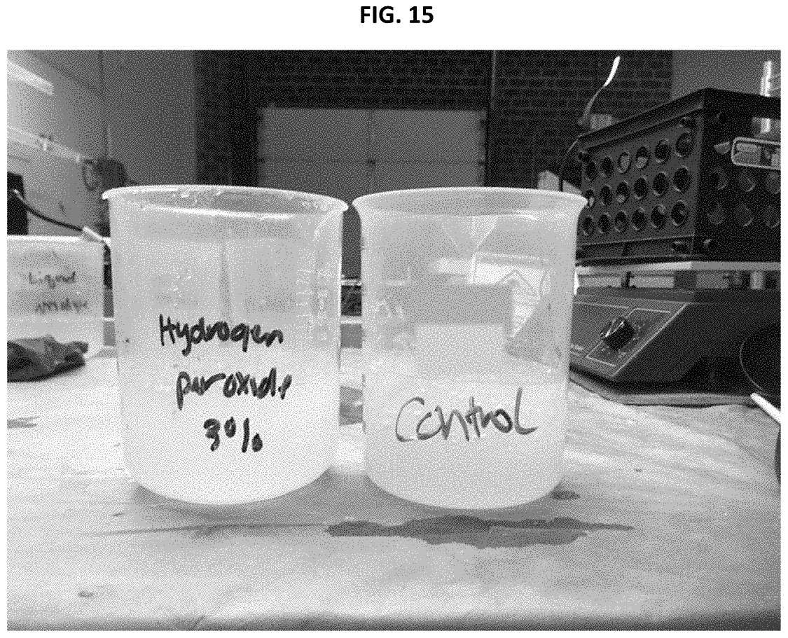

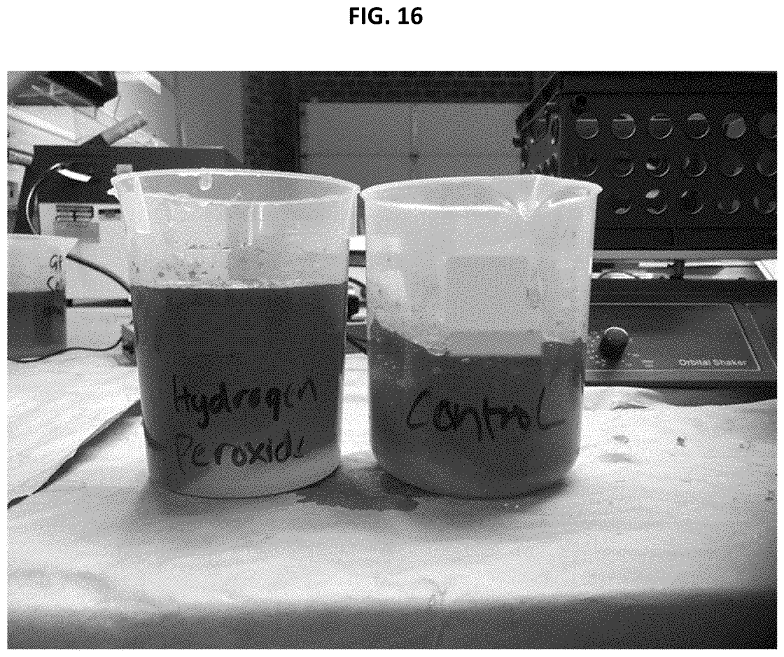

A test sample was prepared by swelling 15.0 grams of a modified starch-based biopolymer having a centrifuge retention capacity (CRC) of at least about 12 g/g or an absorbency under load (AUL) at 0.7 psi of at least about 8 g/g in a saline solution and an amylose content of less than about 85% w/w to about 90% using a 0.9 wt. % saline solution to mimic swelling of the SAM in an absorbent core of a diaper. Approximately 300 milliliters of water was added and 15.0 milliliters of hydrogen peroxide (3 wt. %) was added. A control sample was also prepared by swelling 15.0 grams of the modified starch-based biopolymer utilized above to about 90% using a 0.9 wt. % saline solution to mimic swelling of the SAM in an absorbent core of a diaper. Approximately 300 milliliters of water was added to the control sample. The test sample and the control sample were stirred and visually analyzed for 30 minutes, then left to sit for approximately 6 hours. After sitting for approximately 6 hours, the samples were each filtered through 150-micron mesh sieves. FIG. 15 is a photograph of the residue of the samples after the samples were filtered through the 360-micron mesh sieves. The test sample is to the left of the control sample in FIG. 15. The samples were then transferred to approximately 2000 milliliters of water and were allowed to sit overnight, or for approximately 18 hours to provide time for any reswelling to occur. The samples were then filtered again through 150-micron mesh sieves. FIG. 16 is a photograph of the residue of the samples after the samples were allowed to sit for approximately 18 hours and filtered the second time through the 150-micron mesh sieves. The test sample is to the left of the control sample in FIG. 16. As is evident from FIG. 16, the residue of the test sample which was treated with hydrogen peroxide (3 wt. %) was much less viscous than the control sample after soaking in water for approximately 18 hours. All prior steps were all performed at room temperature under standard pressure.

### EXAMPLE 6: MODIFIED STARCH-BASED BIOPOLYMER TREATED WITH HYDROGEN PEROXIDE (35 WT. %)

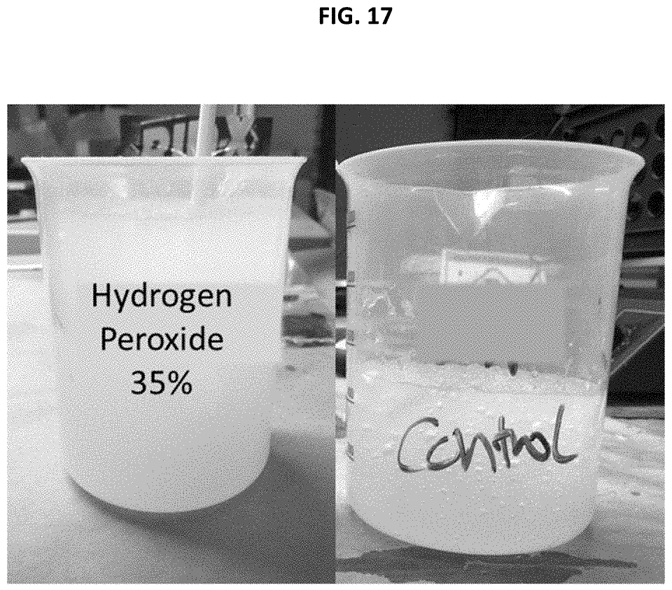

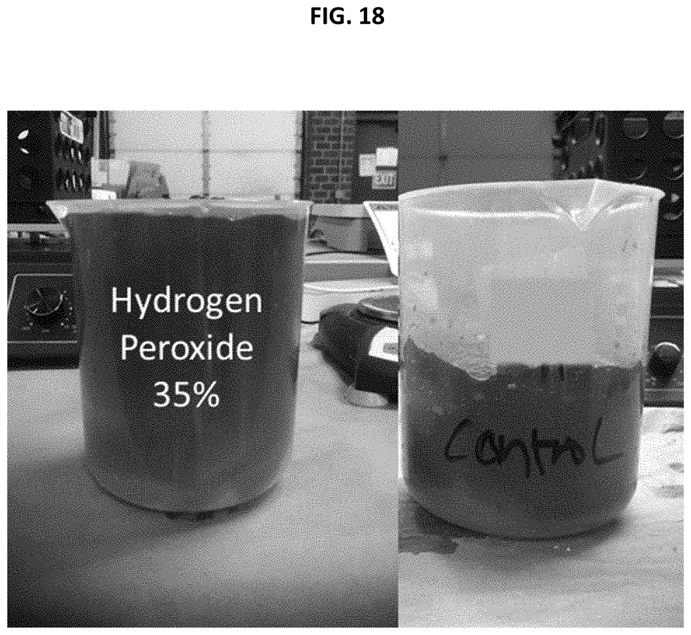

A test sample was prepared by swelling 15.0 grams of a modified starch-based biopolymer having a centrifuge retention capacity (CRC) of at least about 12 g/g or an absorbency under load (AUL) at 0.7 psi of at least about 8 g/g in a saline solution and an amylose content of less than about 85% w/w to about 90% using a 0.9 wt. % saline solution to mimic swelling of the SAM in an absorbent core of a diaper. Approximately 300 milliliters of water was added and 15.0 milliliters of hydrogen peroxide (35 wt. %) was added. A control sample was also prepared by swelling 15.0 grams of the modified starch-based biopolymer utilized above to about 90% using a 0.9 wt. % saline solution to mimic swelling of the SAM in an absorbent core of a diaper. Approximately 300 milliliters of water was added to the control sample. The test sample and the control sample were stirred and visually analyzed for 30 minutes, then left to sit for approximately 6 hours. FIG. 17 is a photograph of the residue of the samples after the samples sat for approximately 6 hours. The test sample is to the left of the control sample in FIG. 17. Interestingly, the 35 wt. % hydrogen peroxide solution did not meaningfully lower the viscosity of the SAM. Accordingly, solutions including less than 35 wt. % hydrogen peroxide, or solutions that do not include more than 35 wt. % hydrogen peroxide, are preferably not utilized in the present invention. The samples were then transferred to approximately 2000 milliliters of water and were allowed to sit overnight, or for approximately 18 hours to provide time for any reswelling to occur. The samples were then filtered through 150-micron mesh sieves. FIG. 18 is a photograph of the residue of the samples after the samples were allowed to sit for approximately 18 hours and filtered the second time through the 150-micron mesh sieves. The test sample is to the left of the control sample in FIG. 18. As is evident from FIG. 18, the residue of the test sample which was treated with hydrogen peroxide (35 wt. %) was less viscous than the control sample after soaking in water for approximately 18 hours. All prior steps were all performed at room temperature under standard pressure.

### EXAMPLE 7: MODIFIED STARCH-BASED BIOPOLYMER TREATED WITH SODIUM HYPOCHLORITE

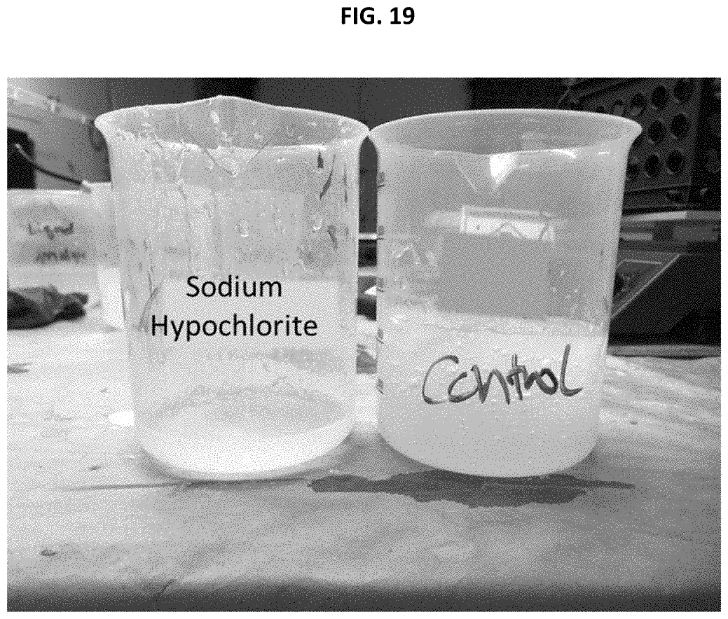

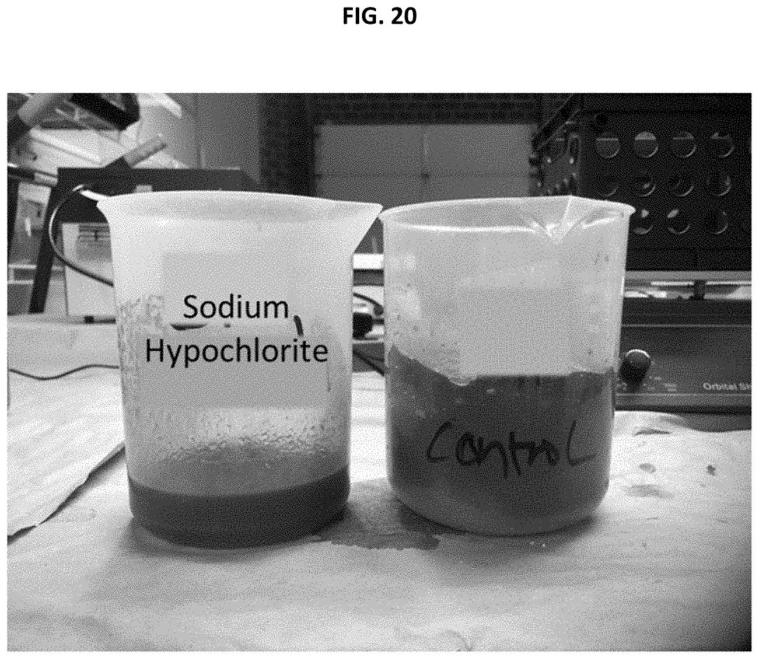

A test sample was prepared by swelling 15.0 grams of a modified starch-based biopolymer having a centrifuge retention capacity (CRC) of at least about 12 g/g or an absorbency under load (AUL) at 0.7 psi of at least about 8 g/g in a saline solution and an amylose content of less than about 85% w/w to about 90% using a 0.9 wt. % saline solution to mimic swelling of the SAM in an absorbent core of a diaper. Approximately 300 milliliters of water was added and 15.0 milliliters of sodium hypochlorite (6 wt. %) was added. A control sample was also prepared by swelling 15.0 grams of the modified starch-based biopolymer utilized above to about 90% using a 0.9 wt. % saline solution to mimic swelling of the SAM in an absorbent core of a diaper. Approximately 300 milliliters of water was added to the control sample. The test sample and the control sample were stirred and visually analyzed for 30 minutes, then left to sit for approximately 6 hours. After sitting for approximately 6 hours, the samples were each filtered through 150-micron mesh sieves. FIG. 19 is a photograph of the residue of the samples after the samples were filtered through the 150-micron mesh sieves. The test sample is to the left of the control sample in FIG. 19. The samples were then transferred to approximately 2000 milliliters of water and were allowed to sit overnight, or for approximately 18 hours to provide time for any reswelling to occur. The samples were then filtered again through 150-micron mesh sieves. FIG. 20 is a photograph of the residue of the samples after the samples were allowed to sit for approximately 18 hours and filtered the second time through the 150-micron mesh sieves. The test sample is to the left of the control sample in FIG. 20. As is evident from FIG. 20, the residue of the test sample which was treated with sodium hypochlorite (6 wt. %) was much less viscous than the control sample after soaking in water for approximately 18 hours. All prior steps were all performed at room temperature under standard pressure.

### EXAMPLE 8: MODIFIED STARCH-BASED BIOPOLYMER TREATED WITH ENZYME MIXTURE

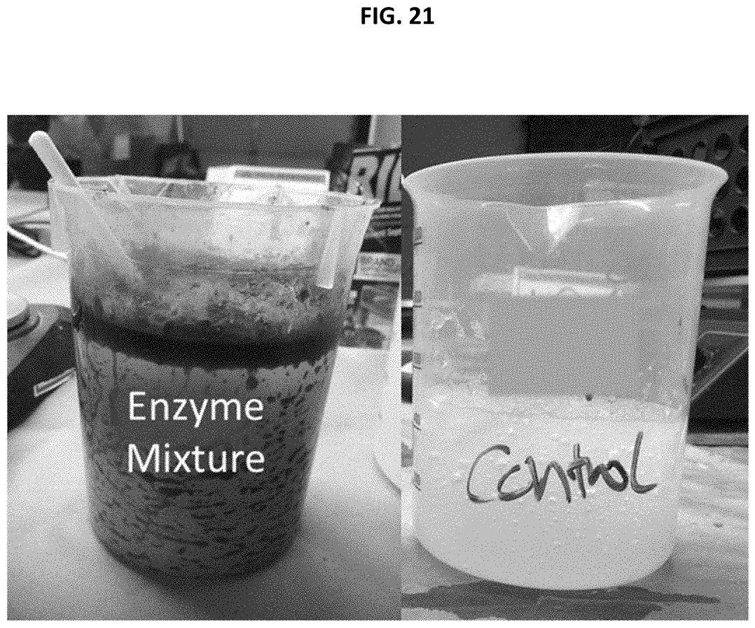

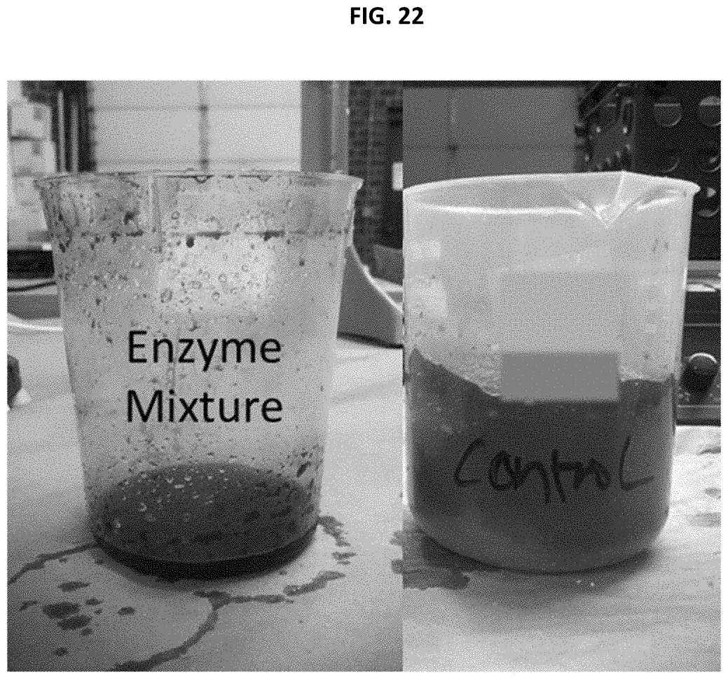

A test sample was prepared by swelling 15.0 grams of a modified starch-based biopolymer having a centrifuge retention capacity (CRC) of at least about 12 g/g or an absorbency under load (AUL) at 0.7 psi of at least about 8 g/g in a saline solution and an amylose content of less than about 85% w/w to about 90% using a 0.9 wt. % saline solution to mimic swelling of the SAM in an absorbent core of a diaper. Approximately 300 milliliters of water was added and 15.0 grams of an enzyme mixture including amylase, lipase, cellulase, and protease were added. In other embodiments of the present invention, other septic tank treatment agents which include amylase, lipase, cellulase, and/or protease are operable to be utilized to degrade or restructure the SAM. A control sample was also prepared by swelling 15.0 grams of the modified starch-based biopolymer utilized above to about 90% using a 0.9 wt. % saline solution to mimic swelling of the SAM in an absorbent core of a diaper. Approximately 300 milliliters of water was added to the control sample. The test sample and the control sample were stirred and visually analyzed for 30 minutes, then left to sit for approximately 6 hours. After sitting for approximately 6 hours, the samples were each filtered through 350-micron mesh sieves. FIG. 21 is a photograph of the samples after the samples were filtered through the 350-micron mesh sieves. The test sample is to the left of the control sample in FIG. 21. The samples were then transferred to approximately 2000 milliliters of water and were allowed to sit overnight, or for approximately 18 hours to provide time for any reswelling to occur. FIG. 22 is a photograph of the samples after the samples were allowed to sit for approximately 18 hours. The test sample is to the left of the control sample in FIG. 22. As is evident from FIG. 22, the test sample which was treated with the enzyme mixture was much less viscous than the control sample after soaking in water for approximately 18 hours. All prior steps were all performed at room temperature under standard pressure.

### EXAMPLE 9: CROSSLINKED SODIUM POLYACRYLATE SUPERABSORBENT POLYMER SAMPLES TREATED WITH LIQUID AMYLASE, SOLID AMYLASE, PROBIOTICS, HYDROGEN PEROXIDE, MAGNESIUM SULFATE, SODIUM HYPOCHLORITE, AND POTASSIUM PEROXYMONOSULFATE

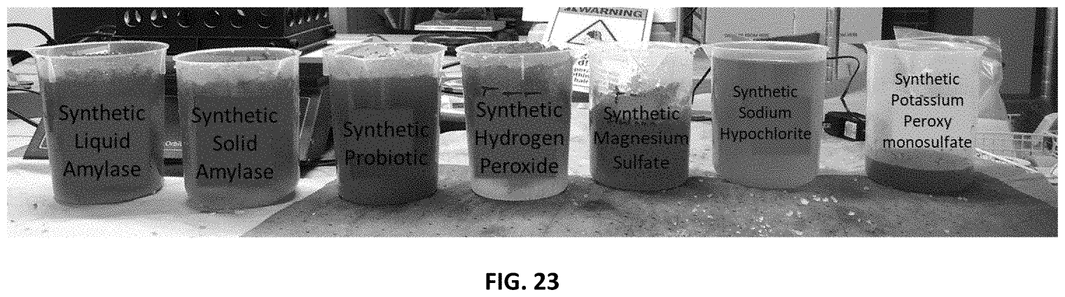

Test samples were prepared by swelling 15.0 grams of crosslinked sodium polyacrylate superabsorbent polymer, to about 90% using a 0.9 wt. % saline solution to mimic swelling of the SAM in an absorbent core of a diaper. The crosslinked sodium polyacrylate superabsorbent polymer has a Free Swell Capacity of 57.1 g/g, a Centrifuge Retention Capacity of 31.9 g/g, and an Absorption Under Load of 15.7 psi in 0.9 wt % NaCl. Approximately 300 milliliters of water was added to each sample. Additionally, approximately 15.0 milliliters of amylase was added to a first test sample, approximately 15.0 grams of solid amylase was added to a second test sample, 15 capsules of probiotic including 75,000 DU of amylase, 360,000 HUT of protease, 18,750 FIP of lipase, and 7,500 CU of cellulase were added to a third test sample, approximately 15.0 milliliters of hydrogen peroxide (3 wt. %) was added to a fourth test sample, approximately 15.0 grams of magnesium sulfate was added to a fifth test sample, approximately 15.0 milliliters of sodium hypochlorite (6 wt. %) was added to a sixth test sample, and approximately 15.0 grams of a multipurpose spa cleaner comprising 31% potassium peroxymonosulfate w/w was added to a seventh test sample. The test samples were stirred and visually analyzed for 30 minutes, then left to sit for approximately 6 hours. After sitting for approximately 6 hours, the samples were each filtered through 150-micron mesh sieves. The samples were then transferred to approximately 2000 milliliters of water and were allowed to sit overnight, or for approximately 18 hours to provide time for any reswelling to occur. FIG. 23 is a photograph of the samples after the samples were allowed to sit for approximately 18 hours. From left to right, the test samples pictured are the first test sample which was treated with liquid amylase, the second test sample which was treated with solid amylase, the third test sample which was treated with probiotic including 75,000 DU of amylase, 360,000 HUT of protease, 18,750 FIP of lipase, and 7,500 CU of cellulase, the fourth test sample which was treated with hydrogen peroxide, the fifth test sample which was treated with magnesium sulfate, the sixth test sample which was treated with sodium hypochlorite, and the seventh test sample which was treated with potassium peroxymonosulfate. As is evident from FIG. 23, a multipurpose spa cleaner comprising 31% potassium peroxymonosulfate w/w meaningfully changed the viscosity of the sodium salt of crosslinked polyacrylic acid swollen in the 0.9 wt. % saline solution. Additionally, as is evident from FIG. 23, solid amylase, liquid amylase, and probiotics also did not meaningfully change the viscosity of the sodium salt of crosslinked polyacrylic acid swollen in the 0.9 wt. % saline solution. The multipurpose spa cleaner comprising 31% potassium peroxymonosulfate w/w was most effective of these chemical agents in reducing viscosity of the liquid including the SAM. All prior steps were all performed at room temperature under standard pressure.

### EXAMPLE 10: CROSSLINKED SODIUM POLYACRYLATE SUPERABSORBENT POLYMER SAMPLES TREATED WITH SODIUM HYPOCHLORITE AND POTASSIUM PEROXYMONOSULFATE

Test samples were prepared by swelling 15.0 grams of crosslinked sodium polyacrylate superabsorbent polymer to about 90% using a 0.9 wt. % saline solution to mimic swelling of the SAM in an absorbent core of a diaper. The crosslinked sodium polyacrylate superabsorbent polymer has a Free Swell Capacity of 57.1 g/g, a Centrifuge Retention Capacity of 31.9 g/g, and an Absorption Under Load of 15.7 psi in 0.9 wt % NaCl. Approximately 300 milliliters of water was added to each sample. Additionally, approximately 15.0 milliliters of amylase was added to a first test sample, approximately 15.0 grams of solid amylase was added to a second test sample, 15 capsules of probiotic including 75,000 DU of amylase, 360,000 HUT of protease, 18,750 FIP of lipase, and 7,500 CU of cellulase were added to a third test sample, approximately 15.0 milliliters of hydrogen peroxide (3 wt. %) was added to a fourth test sample, approximately 15.0 grams of magnesium sulfate was added to a fifth test sample, approximately 15.0 milliliters of sodium hypochlorite (6 wt. %) was added to a sixth test sample, and approximately 15.0 grams of a multipurpose spa cleaner comprising 31% potassium peroxymonosulfate w/w was added to a seventh test sample. The test samples were stirred and visually analyzed for 30 minutes, then left to sit for approximately 6 hours. After sitting for approximately 6 hours, the samples were each filtered through 150-micron mesh sieves. The samples were then transferred to approximately 2000 milliliters of water and were allowed to sit overnight, or for approximately 18 hours to provide time for any reswelling to occur. FIG. 23 is a photograph of the samples after the samples were allowed to sit for approximately 18 hours. The test sample is to the left of the control sample in FIG. 23. As is evident from FIG. 23, a multipurpose spa cleaner comprising 31% potassium peroxymonosulfate w/w changed the viscosity of the sodium salt of crosslinked polyacrylic acid swollen in the 0.9 wt. % saline solution such that sodium polyacrylate superabsorbent polymer degraded to the extent that superabsorbent properties were no longer present. Additionally, as is evident from FIG. 23, solid amylase, liquid amylase, and probiotics also did not meaningfully change the viscosity of the sodium salt of crosslinked polyacrylic acid swollen in the 0.9 wt. % saline solution. All prior steps were all performed at room temperature under standard pressure.

### EXAMPLE 11: MODIFIED STARCH-BASED BIOPOLYMER TREATED WITH VARIOUS RATIOS OF SODIUM HYPOCHLORITE

Three test samples were prepared by swelling 15.0 grams of a modified starch-based biopolymer having a centrifuge retention capacity (CRC) of at least about 12 g/g or an absorbency under load (AUL) at 0.7 psi of at least about 8 g/g in a saline solution and an amylose content of less than about 85% w/w using water. Approximately 1500 milliliters of water was added to each test sample. Then 15.0 milliliters of sodium hypochlorite (6 wt. %) was added to the first sample, 45.0 milliliters of sodium hypochlorite (6 wt. %) was added to the second sample, and 75.0 milliliters of sodium hypochlorite (6 wt. %) was added to the third sample. Two control samples were also prepared. The first control sample was 1500 milliliters of water. The second control sample was prepared by swelling 15.0 grams of the modified starch-based biopolymer utilized above in 1500 milliliters of water. The viscosity of the test samples and control samples were measured after 5 minutes, 15 minutes, 30 minutes and 2 hours using a 61 spindle or 62 spindle. Specifically, the viscosity of the second control sample was measured using a 62 spindle while all other samples were measured using a 61 spindle. The weight of the residue was also measured 2 hours after filtering each test sample and control sample through a mesh sieve. The control samples and the second and third test samples were filtered using a sieve with a mesh size of less than 500 microns and the first test sample was filtered using a sieve with a mesh size of less than 500 microns. The residue from the test samples and control samples were then added to approximately 2300 milliliters of water and left to sit overnight for approximately 18 hours. The test samples and control samples were again filtered through a sieve with a mesh size of less than 500 microns and the weight of the residue for each sample was measured. All prior steps were all performed at room temperature under standard pressure. Table 2 below includes the viscosity measurements and weights of the residues.

TABLE 2
Viscosity Measurements and Residue Weights of Test Samples and Control Samples

| | | | | | | Weight of |
|---|---|---|---|---|---|---|
| | | | | | Weight of | Residue |
| | Viscosity | Viscosity | Viscosity | Viscosity | Residue | After |
| | 5 min. | 15 min. | 30 min. | 2 hours | After | Additional |
| Sample | (cP) | (cP) | (cP) | (cP) | 2 Hours (g) | 18 Hours (g) |
| First Control | 4.65 | 4.71 | 4.7 | 4.68 | NA | NA |
| Sample (water) | | | | | | |
| Second Control | 3620 | 3615 | 3637 | 3634 | 1515 | 1650 |
| Sample (water + | | | | | | |
| 15 g modified | | | | | | |
| starch-based | | | | | | |
| biopolymer) | | | | | | |
| First Test | 334 | 340 | 344 | 8.46 | 1350 | 1238 |
| Sample (water + | | | | | | |
| 15 g modified | | | | | | |
| starch-based | | | | | | |
| biopolymer + | | | | | | |
| 15 mL sodium | | | | | | |
| hypochlorite) | | | | | | |
| Second Test | 172 | 28.2 | 11.17 | 8.46 | 25 | 0 |
| Sample (water + | | | | | | |
| 15 g modified | | | | | | |
| starch-based | | | | | | |
| biopolymer + | | | | | | |
| 45 mL sodium | | | | | | |
| hypochlorite) | | | | | | |
| Third Test | 11.66 | 8.3 | 7.78 | 7.62 | 0 | 0 |
| Sample (water + | | | | | | |
| 15 g modified | | | | | | |
| starch-based | | | | | | |
| biopolymer + | | | | | | |
| 75 mL sodium | | | | | | |
| hypochlorite) | | | | | | |

Notably, the third test sample did not include any particles larger than 150 microns after 2 hours of treatment with the sodium hypochlorite. Additionally, the second test sample did not include any particles larger than 150 microns after resting overnight. This indicates that no SAM remained in the sample, since the SAM has unswollen particle sizes between 150 microns and 850 microns. Notably, swollen particle sizes of the SAM range from approximately 470 microns to 2,600 microns, or larger.

### EXAMPLE 12: FLUSHABILITY OF SODIUM POLYACRYLATE SAM & BIODEGRADABLE STARCH-BASED SAM

Various tests were conducted to compare the flushability of a synthetic petroleum derived sodium polyacrylate SAM to the flushability of a biodegradable starch-based SAM. The sodium polyacrylate SAM is not biodegradable and has a FSC of 57 g/g, a CRC of 32 g/g, and an AAP of 16 g/g in saline. The starch-based SAM is biodegradable and has a FSC of 34 g/g, a CRC of 21 g/g, and an AAP of 8 g/g in saline. The starch-based SAM has particle sizes between 150 microns to 850 microns.

The tests were conducted to mimic conditions at ¾ scale for a size 4 diaper. A size 4 diaper typically includes 12 g of SAM, with 200 mL of saline being added to mimic a typical insult for a diaper. One liter of tap water is then added to mimic a low flush. As the present examples were run at ¾ scale for a size 4 diaper, 150 mL of saline was added to 9 g of SAM to mimic a typical insult, and the SAM was allowed to swell completely. Then 750 mL of tap water was added and the mixture was stirred to mimic a low flush. The viscosity of the solution at 5 minutes, 30 minutes, 4 hours, and 24 hours after the addition of the tap water was then measured. Additionally, 100 mL of the solution was poured through funnels with different diameter openings (1 cm, 1.5 cm, and 2.2 cm), and the amount of flow through the funnels over 30 seconds was measured. One hundred (100) mL of the solution was also poured through a 1 mm screen 1 hour after the addition of the tap water and 4 hours after addition of the tap water, and the volume of solution that flowed through the screen was measured. Finally, 24 hours after the addition of the tap water, all the remaining solution was poured into a large funnel that feeds a 2″ pipe at a 14% slope (to horizontal), with the end of the pipe feeding into a beaker with a screen over the beaker. Any solids remaining in the pipe were recorded, as well as the presence of solids on the screen and the volume of liquid that flowed through the screen into the beaker. If any solids remained on the pipe, an additional 500 mL of water was added, and it was noted if the additional 500 mL of water forced the rest of the solids in the pipe to flow to the screen/beaker. The results of these tests are presented in Table 3 below.

TABLE 3
Viscosity Measurements, Amount of Flow Through Different Size
Funnels, Amount of Flow Through Screen, and Solids in Pipe
or on Screen for Sodium Polyacrylate SAM & Starch-Based SAM

| | | | Sodium | Starch-Based |
|---|---|---|---|---|
| | | | Polyacrylate | SAM |
| Viscosity | 5 | min | 8300 | 83 |
| (centipoise) | 30 | min | 7000 | 58 |
| at different | 4 | hours | 7000 | 72 |
| times | 24 | hours | 3600 | 82 |
| 30 min Funnel | 1 | cm diameter | 70 mL | 100 mL |
| flow (out of | 1.5 | cm diameter | 70 mL | N/A |
| 100 ml) | 2.2 | cm diameter | 100 mL | N/A |
| Screen Test | 1.5 | hour | 10 mL | 50 mL |
| Flow out of | 4 | hours | 10 mL | 60 mL |
| 100 mL | | | | |

| | | | |
|---|---|---|---|
| 24 hr pipe | After Pour | Solids in | All |
| | | the pipe | cleared |
| | After adding | All | N/A |
| | 500 mL water | cleared | |
| Screen Test | Solids on | Yes | Yes |
| After Pipe | the Screen | | |
| | after pour | | |
| | through Pipe | | |

### EXAMPLE 13: FLUSHABILITY OF SODIUM POLYACRYLATE SAM & BIODEGRADABLE STARCH-BASED SAM AFTER TREATMENT WITH GEL BREAKERS

Solutions were prepared according to the same tests described in Example 12, except that a variety of different gel breakers were added to the mixture with the 750 mL of water and stirred. Specifically, in separate experiments, 18 mL of sodium hypochlorite (chlorine bleach), 27 mL of Septic Treatment with Enzymes, 9 g of solid sodium percarbonate, 18 g of quick dissolving solid sodium tetraborate, 27 g of septic system treatment powder, and 9 g of enzymatic detergent was added to the mixture with the 750 mL of tap water and stirred. The gel breakers were added to both the swollen sodium polyacrylate and biodegradable starch-based SAMs. The same tests as in Example 12 were then conducted; the results are presented in Tables 4A & 4B below.

TABLE 4A
Viscosity Measurements, Amount of Flow Through Different Size Funnels, Amount of Flow Through Screen,
and Solids in Pipe or on Screen for Sodium Polyacrylate SAM After Treatment with Gel Breakers

| | | | Gel Breaker | | | | | |
|---|---|---|---|---|---|---|---|---|
| | | | | Septic | | | | |
| | | | | Treatment | | | Septic | |
| | | | Sodium | with | Sodium | Sodium | System | Enzymatic |
| | | | Hypochlorite | Enzymes | Percarbonate | Tetraborate | Powder | Detergent |
| Viscosity | 5 | min | 39 | 3500 | 40 | 45 | 38 | 612 |
| (centipoise) | 30 | min | 179 | 2400 | 27 | 125 | 113 | 97 |
| at different | 4 | hours | 4100 | 1600 | 56 | 166 | 170 | N/A |
| times | 24 | hours | 162 | 1200 | 162 | 380 | 69 | 343 |
| 30 min Funnel | 1 | cm | 100 mL | 70 mL | 100 mL | 100 mL | 95 mL | 100 mL |
| flow (out of | 1.5 | cm | N/A | 95 mL | N/A | N/A | N/A | N/A |
| 100 mL) | 2.2 | cm | N/A | N/A | N/A | N/A | N/A | N/A |
| Screen Test | 1.5 | hour | 20 mL | 10 mL | 45 mL | 60 mL | N/A | N/A |
| Flow out of | 4 | hours | 15 mL | 10 mL | 60 mL | 60 mL | 60 mL | 35 mL |
| 100 mL | | | | | | | | |

| | | | | | | | |
|---|---|---|---|---|---|---|---|
| 24 hr pipe | After Pour | Cleared | Solids | Cleared | Solids | Solids | Cleared |
| | | | in Pipe | | in pipe | in pipe | |
| | After adding | N/A | Cleared | N/A | Cleared | Cleared | N/A |
| | 500 mL water | | | | | | |
| Screen Test | Solids on | Yes | Yes | Yes | Yes | Yes | Yes |
| After Pipe | the Screen | | | | | | |
| | after pipe | | | | | | |

TABLE 4B
Viscosity Measurements, Amount of Flow Through Different Size Funnels, Amount of Flow Through Screen,
and Solids in Pipe or on Screen for Biodegradable Starch-Based SAM After Treatment with Gel Breakers

| | | | Gel Breaker | | | | | |
|---|---|---|---|---|---|---|---|---|
| | | | | Septic | | | | |
| | | | | Treatment | | | Septic | |
| | | | Sodium | with | Sodium | Sodium | System | Enzymatic |
| | | | Hypochlorite | Enzymes | Percarbonate | Tetraborate | Powder | Detergent |
| Viscosity | 5 | min | 45 | 55 | 11 | 52 | 26 | 27 |
| (centipoise) | 30 | min | 23 | 48 | 9 | 46 | 73 | 95 |
| at different | 4 | hours | 11 | 31 | 8 | 174 | 64 | N/A |
| times | 24 | hours | 9 | 66 | 10 | 15 | 392 | 8 |
| 30 min Funnel | 1 | cm | 100 mL | 100 mL | 100 mL | 100 mL | 100 mL | 100 mL |
| flow (out of | 1.5 | cm | N/A | N/A | N/A | N/A | N/A | N/A |
| 100 mL) | 2.2 | cm | N/A | N/A | N/A | N/A | N/A | N/A |
| Screen Test | 1.5 | hour | 100 mL | 60 mL | 100 mL | 65 mL | 75 mL | N/A |
| Flow out of | 4 | hours | 100 mL | 55 mL | 100 mL | 95 mL | 80 mL | 100 mL |
| 100 mL | | | | | | | | |

| | | | | | | | |
|---|---|---|---|---|---|---|---|
| 24 hr pipe | After Pour | All | All | All | All | Solids in | All |
| | | cleared | cleared | cleared | cleared | the pipe | cleared |
| | After adding | N/A | N/A | N/A | N/A | All | N/A |
| | 500 mL water | | | | | cleared | |
| Screen Test | Solids on | No | Yes | No | No | Yes | No |
| After Pipe | the Screen | | | | | | |
| | after pipe | | | | | | |

The tables above show relative performance of the different gel breakers for each SAM type. For the sodium polyacrylate SAM, treatment with the gel breaker caused some reduced swelling potential for the SAM, as evidenced by the test data above after treatment with sodium hypochlorite, sodium percarbonate, and enzymatic detergent. However, no gel breaker completely removes the swelling potential for the sodium polyacrylate SAM, as evidenced by the final screen pour test, in which the sodium polyacrylate SAM does not fit through the 1 mm openings of the screen. This indicates that the sodium polyacrylate SAM is not substantially or completely degraded by the gel breakers and the sodium polyacrylate SAM would still clog standard piping if the sodium polyacrylate SAM were treated with the gel breakers and flushed down a standard toilet. Notably, almost all of the starch-based SAMs treated with gel breakers were degraded such that the treated starch-based SAMs were operable to flow through the 1 mm screen after 24 hours. This indicates that the starch-based SAMs do not clog standard piping if the starch-based SAMs are treated with the sodium hypochlorite, septic treatment with enzymes, sodium percarbonate, sodium tetraborate, or enzymatic detergent. Additionally, the solids in the pipe observed after treatment with the septic system powder was undissolved septic system powder and did not include swollen SAM.

For the remaining sodium polyacrylate SAM which did not pass through the screen, 100 mL of tap water was poured over the sodium polyacrylate SAM three times. The filtrate was then added to a fresh beaker with additional tap water sufficient to create free swell conditions and allowed to sit for 30 minutes. The sodium polyacrylate SAM reswelled to approximately the original reswell size. Significantly, the fact that the sodium polyacrylate SAM reswelled when exposed to these conditions indicates that the sodium polyacrylate SAM was not degraded by the gel breaker, whereas the starch-based SAM was degraded by the gel breaker and the swelling properties of the starch-based SAM were not temporarily affected by solution.

### EXAMPLE 14. FLUSHABILITY TESTING OF ABSORBENT CORES (SAM AND FLUFF) WHERE ENZYMATIC DETERGENT IS ADDED TO THE ABSORBENT CORE

Various tests were conducted to compare the flushability of an absorbent core including a synthetic petroleum derived sodium polyacrylate SAM to the flushability of an absorbent core including a biodegradable starch-based SAM. The sodium polyacrylate SAM is not biodegradable and has a FSC of 58 g/g, CRC of 34 g/g, and an AAP of 22 g/g, in saline. The starch-based SAM is biodegradable and has a FSC of 33 g/g, a CRC of 19 g/g, and an AAP of 9 g/g in saline. The sodium polyacrylate SAM is a different sodium polyacrylate SAM than the sodium polyacrylate SAM utilized in Examples 12-13.

The tests were conducted to mimic conditions at ½ scale for a size 4 diaper. The absorbent core of a size 4 diaper typically includes 12 g of SAM, with the remainder of the absorbent core being fluff, such as wood or paper fiber that has been fluffed. Typically, the absorbent core includes approximately 50% to 60% SAM. Absorbent cores between 21 to 22 g were constructed with the SAMs and fluff using a lab core former, and pressed to a density of between 0.18 to 0.20 g/cm3. These cores were cut in half and 7 g of solid enzymatic detergent was added to a first half of each core at the top layer. The second half of the core was used in Example 15. One hundred and ten (110) mL of saline was added to each half of each core to mimic a typical insult for a diaper. Then 275 mL of tap water was added and the mixture was stirred to mimic a low flush of the absorbent core.

The viscosity of the solution was then measured 5 minutes and 15 minutes after the addition of the 275 mL of tap water. Then 100 additional mL of water was added 20 minutes before the addition of the 275 mL of tap water and stirring of the mixture. The viscosity was measured after the addition of the 100 mL of water, and then 45 minutes and 60 minutes after the addition of the 275 mL of tap water and stirring of the mixture. Compared to Example 13, the fluff in the solution affects the viscosity measurement. Ninety (90) minutes after the addition of the 275 mL of tap water and stirring of the mixture, the solution was poured into a large funnel that feeds a 2″ pipe at a 14% slope (to horizontal), with the end of the pipe feeding into a beaker with a screen over the beaker. Any solids remaining in the pipe were recorded, as well as the presence of solids on the screen and the volume of liquid that flowed through the screen into the beaker. Most of the fluff was observed to be left on the screen. If any solids remained on the pipe, an additional 500 mL of water was added, and it was noted if the additional 500 mL of water forced the rest of the solids in the pipe to flow to the screen/beaker. Table 5 illustrates the results of these tests.

TABLE 5
Viscosity Measurements, Solids on Pipe After Pour, and
Volume of Solution which Flowed through the Screen
for Sodium Polyacrylate SAM & Biodegradable Starch-
Based SAM After Treatment with Enzymatic Detergent

| | | | Sodium | Starch-Based |
|---|---|---|---|---|
| | | | Polyacrylate | SAM |
| Viscosity | 5 | min | 5300 | 2800 |
| (centipoise) | 15 | min | 6900 | 2500 |
| at different | 20 min, 100 mL water | | 2700 | 820 |
| times | was added before | | | |
| | measurement at 20 min | | | |
| | 45 | min | 2600 | 674 |
| | 60 | min | 2500 | 489 |

| | | | |
|---|---|---|---|
| 1.5 hr pipe | After Pour | Solids in | All |
| | | the Pipe | cleared |
| | After adding | All | N/A |
| | 500 mL water | cleared | |
| Volume Through | Solution Flowing | 100 mL | 400 mL |
| the Screen | Through The screen | | |
| | pour of 485 mL | | |

Notably, the results show that the starch-based SAM degraded more than the sodium polyacrylate SAM, as evidenced by the lack of solids in the pipe and the volume of the solution which flowed through the screen. While both SAMs left fluff on the screen, a noticeable amount of swollen sodium polyacrylate remained on the screen because it was not degraded by the detergent.

### EXAMPLE 15. FLUSHABILITY TESTING OF ABSORBENT CORES (SAM AND FLUFF) WHERE SODIUM PERCARBONATE IS ADDED TO THE ABSORBENT CORE

Cores were formed according to the method in Example 14, except that 7 g of sodium percarbonate was added to the top layer of the first half of each core instead of 7 g of solid enzymatic detergent.

The viscosity of the solution was then measured 1 minute, 5 minutes, 10 minutes, and 15 minutes after the addition of the 275 mL of tap water. Thirty (30) minutes after the addition of the 275 mL of tap water and stirring of the mixture, the solution was poured into a large funnel that feeds a 2″ pipe at a 14% slope (to horizontal), with the end of the pipe feeding into a beaker with a screen over the beaker. Any solids remaining in the pipe were recorded, as well as the presence of solids on the screen and the volume of liquid that flowed through the screen into the beaker. Most of the fluff was observed to be left on the screen. If any solids remained on the pipe, 500 mL of water was added, and it was noted if the 500 mL of water forced the rest of the solids in the pipe to flow to the screen/beaker. Table 6 illustrates the results of these tests.

TABLE 6
Viscosity Measurements, Solids on Pipe After Pour, and
Volume of Solution which Flowed through the Screen
for Sodium Polyacrylate SAM & Biodegradable Starch-
Based SAM After Treatment with Sodium Percarbonate

| | | Sodium | Starch-Based |
|---|---|---|---|
| | | Polyacrylate | SAM |
| Viscosity | 1 min | 222 | 152 |
| (centipoise) | 5 min | 213 | 175 |
| at different | 15 min | 1600 | 2200 |
| times | | | |
| 30 min Pipe | After Pour | Solids in | All |
| | | the pipe | cleared |
| | After adding | All cleared | N/A |
| | 500 mL water | | |
| Volume Through | Solution Flowing | 250 mL | 500 mL |
| the Screen | Through The screen | | |

Notably, the results show that the starch-based SAM degraded more than the sodium polyacrylate SAM, as evidenced by the lack of solids in the pipe and the volume of the solution which flowed through the screen. While both SAMs left fluff on the screen, a noticeable amount of swollen sodium polyacrylate remained on the screen because it was not degraded by the detergent.

### EXAMPLE 16. TIME TO BREAK DOWN THE SWOLLEN SAM WITH GEL BREAKERS

This example determines the time it takes to degrade at least 90% of the starch-based SAM of Examples 12-15 using different gel breakers. As the dry particle sizes for the starch-based SAM used in Example 12-15 are between 150 and 850 microns, swollen SAM particles are operable to swell to above sizes of 1 mm. This is due to the high Free Swell Capacity (FSC) of these SAMs. Therefore, a swollen SAM particle will not flow through the 1 mm screen unless its size is dramatically reduced by the gel breaker.

For this example, mixtures of 12 g of the starch-based SAM used in Examples 12-15 were swollen with 200 mL of saline. Then 300 mL of tap water was added along with the gel breaker dissolved in 100 mL of tap water and stirred. Differing amounts of gel breakers were added, which are detailed in the table below. At the various given time intervals, a 100 mL beaker was filled with solution, weighed, and the solution was poured through the screen. The amount of material was then put into the beaker and weighed. The difference was the % that went through the screen.

The time to breakdown at least 90% of the starch-based SAM is shown in the table below at different times with gel breakers. The time to breakdown at least 90% of the starch-based SAM was measured by the amount of solution along with the SAM that flows through the screen.

TABLE 7
Percentages of the Total Solution that Passes through
1 mm Screen After Treatment with Gel Breaker

| | Gel Breaker | | | | |
|---|---|---|---|---|---|
| | | | | | Liquid |
| | Sodium | Sodium | Sodium | Sodium | septic |
| | percarbonate | hypochlorite | hypochlorite | tetraborate | treatment |
| Mixed Time to | Amount of Gel Breaker | | | | |
| Filter (min) | 12 g | 36 mL | 24 mL | 12 g | 12 mL |
| | 58% | 51% | 23% | 23% | 13% |
| 1 | 88% | 79% | 30% | 19% | N/A |
| 2 | 100% | 88% | 53% | 19% | 12% |
| 3 | 98% | 94% | 74% | N/A | N/A |
| 4 | 98% | 96% | 85% | 19% | 11% |
| 5 | 100% | 97% | 93% | 22% | 12% |
| 10 | N/A | N/A | N/A | 22% | 9% |
| 40 | N/A | N/A | N/A | 99% | N/A |
| 16 hrs | N/A | N/A | N/A | N/A | 53% |
| 24 hrs | N/A | N/A | N/A | N/A | 86% |

As illustrated in the table above, three of the gel breakers caused the starch-based SAM to degrade 90% breakdown in 5 minutes or less after the addition of the gel breaker and the tap water. Two gel breakers caused at least 95% of the total solution to pass through the screen in less than 5 minutes after addition of the gel breaker and the tap water. Notably, sodium percarbonate caused at least 98%, and more preferably about 100%, of the total solution to pass through the screen 2 minutes after addition of the gel breaker and the tap water. This indicates that at least 98%, and in some embodiments about approximately 100%, of the starch-based SAM is degraded after 2 minutes of exposure to the sodium hypochlorite. Generally, fast breakdown or degradation of the SAM is best for the initial flushing process.

### EXAMPLE 18. TIME TO BREAK DOWN THE SWOLLEN SAM WITH FASTEST GEL BREAKER

This example determined the fastest time to greater than 90% breakdown or degradation of the starch-based SAM with sodium percarbonate, the gel breaker that degraded the starch-based SAM the fastest in Example 17. In this test 1 gram of starch-based SAM was mixed with 20 mL of saline. Three grams of the sodium percarbonate was added to 30 mL of tap water. Greater than 90% of the SAM passed through the 1 mm screen in 50 seconds. When the sodium percarbonate was added with no additional water, greater than 90% of the SAM passed through the 1 mm screen in 35 seconds.

Based on the examples above, the biodegradable modified starch-based biopolymer was degraded such that the superabsorbent properties were no longer present. As noted above, a level of degradation acceptable for flushing the treated SAM was not seen with the acrylate-based polymer, except when a multipurpose spa cleaner comprising 31% potassium peroxymonosulfate w/w was used as the restructuring agent. Accordingly, both the enzymes and free radical chemicals utilized in the examples with the biodegradable modified starch-based biopolymer were capable of reversing the superabsorbent properties of and further degrading the biodegradable modified starch-based biopolymer, whereas these enzymes and free radical chemicals were not effective in reversing the superabsorbent properties of and degrading the acrylate-based polymer.

The present invention thereby provides for restructuring the soiled or swollen SAM such that the soiled or swollen SAM is transformed into other chemicals and/or undergoes physical change to create particles which have a reduced retention capacity and reduced absorption capacity compared to the unswollen and unsoiled SAM. Notably, the gel blockers or restructuring agents of the present invention are operable to biochemically, chemically, and/or physically degrade the SAM such that it does not swell, or less than 10% of the particles of the SAM swell under free swell conditions compared to an untreated SAM. Bacteria and/or enzymes are also operable to biochemically, chemically, and/or physically degrade the SAM such that it does not swell, or less than 10% of the particles of the SAM swell under free swell conditions compared to an untreated SAM. In other words, the SAM does not reswell after treatment with the restructuring agent or gel breaker under free swell conditions, and the entire SAM is permanently degraded or deactivated. In other embodiments, at least 90% of the particles of the SAM do not reswell after treatment with the restructuring agent or gel breaker under free swell conditions, and the SAM is permanently degraded or deactivated. In other embodiments, at least 95% or at least 98% of the SAM does not reswell after treatment with the restructuring agent or gel breaker under free swell conditions, and the SAM is permanently degraded or deactivated. The deactivation or degradation of the SAM is performed at room temperature under standard pressure. Therefore, the present invention is advantageous over processes for deactivating soiled or swollen SAMs such as those described in U.S. Pat. No. 10,538,878 by inventor Konishi where significantly higher pressure and/or temperature must be utilized.

Additionally, while a-amylase was utilized in the examples above, saccharifying α-amylase, beta-amylase, glucoamylase, mannanase, cellulase, lipase, and pullulanase are also utilized in the present invention for hydrolysis of the starch of the biodegradable modified starch-based biopolymer.

In one embodiment, the present invention utilizes a tank operable to hold water, a restructuring agent which restructures one or more soiled or swollen SAMs to make the soiled or swollen SAMs flushable and treatable by existing standard municipal waste water treatment facilities. As discussed with respect to FIG. 2 above, the restructuring agent is operable to be blended with the SAM in the removable absorbent core of a diaper, or in any other portion of the hygienic article which is being flushed such that the restructuring agent is activated when the removable absorbent core contacts water. The tank is operable to be standalone in one embodiment. Alternatively, the tank is connected to plumbing of a standard toilet or another plumbing access point such that the tank is operable to receive water from the plumbing. In another embodiment, the tank does not require addition of water but only requires addition of a biodegradable hygienic article including a soiled or swollen SAM or a portion of a hygienic article including a soiled or swollen SAM which is biodegradable and a restructuring agent. The tank preferably includes a waste disposal mechanism which is operable to empty into a toilet bowl. Alternatively, the tank contains two separate compartments; one compartment for collecting the flushable absorbent core and a second compartment for collecting the non-flushable hygienic article. Additionally, the first compartment is connected to plumbing of a standard toilet or another plumbing access point. In one embodiment, the second compartment is standalone, and operable to dispose of or recycle the non-flushable hygienic article. Additionally, the tank contains a mechanism that separates the flushable absorbent core from the non-flushable hygienic article. In one embodiment, the mechanism is a physical agitator that physically breaks down the absorbent core so that the absorbent core is easily separated from the hygienic article shell. The mechanism physically agitates the hygienic article such that the absorbent core is exposed to the restructuring agent and water to facilitate the restructuring reaction. Additionally, the mechanism separates the hygienic article shell from the absorbent core into the secondary compartment. In an embodiment in which the tank is standalone, the tank is operable to be manually emptied into a toilet bowl via removal of the lid of the tank and physically dumping the contents of the tank into the toilet bowl. In one embodiment, the tank includes a physical agitation component operable to break down the fibers or fluff of absorbent cores or other hygiene article components placed in the tank. The physical agitation component preferably physically breaks down the hygienic article or agitates the hygienic article such that it is exposed to the restructuring agent and water to facilitate the restructuring reaction. Additionally or alternatively, the tank includes a chopping, cutting, or shredding mechanism or component to chop, cut, or shred fibers of the core. In yet another embodiment, a chemical is added to the tank to break down the fibers of fluff of absorbent cores or other hygiene article components placed in the tank. Preferably, the lid of the tank includes a rubber gasket operable to provide a water-tight and air-tight seal with the base of the tank. This is advantageous in preventing spills from the tank and helps reduce odors coming from the tank. Preferably, the tank includes a stainless-steel shell, which also has the advantage of minimizing odors from the tank.

In one embodiment, the tank includes a heater operable to increase the temperature inside the tank to expedite the restructuring of the soiled or swollen SAM. The heater is preferably operable to increase the temperature of the contents of the tank to up to 140 degrees Fahrenheit. Alternatively, the heater is operable to increase the temperature of the contents of the tank to up to 120 degrees Fahrenheit. The power source for the heater is operable to be a standard plug operable to plug in to a standard wall outlet in one embodiment. Advantageously, by only providing heat that is not hotter than hot water produced by a standard hot water heater, the present invention provides a safe mechanism for expediting restructuring of soiled or swollen SAMs while not introducing a health and safety hazard. As previously mentioned, commercial recycling of diapers often involves deactivation of SAMs using temperatures and pressures that are not safe for an ordinary consumer. Thus, the present invention preferably does not involve heating materials including soiled or swollen SAMs to above 140 degrees Fahrenheit. Alternatively, the present invention does not involve heating materials including soiled or swollen SAMs to above 150 degrees Fahrenheit, above 160 degrees Fahrenheit, above 170 degrees Fahrenheit, above 180 degrees Fahrenheit, above 190 degrees Fahrenheit, or above 200 degrees Fahrenheit. Rather, heat of up to approximately 140 degrees Fahrenheit is utilized in one embodiment of the present invention to accelerate the reaction between soiled or swollen SAMs and restructuring agents.

In another embodiment, the present invention includes a liquid formulation or solid formulation which is operable to be added to a toilet bowl along with a hygienic article or a portion of a hygienic article such as a removable, absorbent core. The liquid formulation or solid formulation includes a restructuring agent which restructures the soiled or swollen SAM included in the hygienic article into chemicals which are operable to be flushed down a standard toilet without clogging the plumbing when flushed or over a period of repeated flushes of the chemicals. In one embodiment, the solid formulation is a powder. Alternatively, the solid formulation is a pill, tablet, or capsule. In one embodiment, a waiting period is required while the hygienic article or portion of the hygienic article including the soiled or swollen SAM and the liquid formulation or solid formulation are in the toilet bowl in order for the soiled or swollen SAM to be restructured by the restructuring agent in the liquid formulation or solid formulation. The waiting period is operable to be between about 2 minutes to about 40 minutes, and more preferably between about 2 minutes to about 5 minutes. In another embodiment, the waiting period is less than about 3 minutes. Catalysts are operable to be included with the restructuring agent, regardless of whether in a solid form, liquid form, gel form, capsule, gel pod, or other form. Alternatively, the hygienic article or portion of the hygienic article is stirred or otherwise physically manipulated to facilitate breakdown of the hygienic article including chemical restructuring or physical transformation of the soiled or swollen SAM. In another embodiment, the liquid formulation or solid formulation includes an agent which is operable to break down fluff, pulp, or other fibers included in the hygienic article or the portion of the hygienic article including the soiled or swollen SAM. By way of example, cellulase and/or amylase are included in the liquid formulation or solid formulation to break down fluff, pulp, and/or other fibers.

In other embodiments, the starch-based SAM of the present invention is operable to be used in applications which incorporate the starch-based SAM into wound dressings, agriculture applications such as soil amendments, hospital pads, food packaging, pet pads, gel ice packs, cold shipping packs, water barrier bags, fracking, etc. The starch-based SAM is also operable to be used in thickeners or rheology modifiers. The gel breakers or degradation agents of the present invention are operable to degrade the starch-based SAM such that the starch-based SAM loses at least 90% of the absorption capacity or swelling capacity. In some embodiments, the degradation agent is incorporated into the structure of a product or article and activated in predetermined conditions including exposure to another chemical, a temperature, deterioration of a physical boundary between the degradation agent and the starch-based SAM, etc.

In one embodiment, the starch-based SAM is utilized in a wound care application. The starch-based SAM is integrated with a swab, a wound fabric, a wound dressing, a wound compress, a wound pad, a bandage, a patch, and/or a stocking. Materials for these products are operable to be woven or nonwoven. The crosslinked, charge-modified biopolymer is operable to be in a powder or foam form when integrated. In one embodiment, an absorbent component of the wound care article includes a nonwoven or airlaid component which includes the starch-based SAM. The starch-based SAM is preferably mixed with wood fluff. The wound care article includes a fluid permeable topsheet and a fluid impermeable backsheet in one embodiment. Adhesives are preferably biobased and biodegradable adhesives, such as starch-based or soy-based adhesives. Preferably, antimicrobial components are also included in the wound care article. Antimicrobial components traditionally have included metals such as silver or copper. However, the antimicrobial component included in wound care articles according to the present invention is preferably biodegradable, such as the biodegradable compositions recited in U.S. Pat. No. 9,555,167, titled “Biocompatible antimicrobial compositions” and issued on Jan. 31, 2017, which is hereby incorporated herein by reference in its entirety. Application of the degradation agent to the starch-based SAM causes the starch-based SAM to degrade such that the starch-based SAM is not operable to swell or reswell under free swell conditions. In one embodiment, the degradation agent is included in a layer of the wound dressing and is operable to be mixed with the starch-based SAM upon degradation of a barrier between the absorbent core including the starch-based SAM or upon exposure to water or another activation agent.

In another example embodiment, the crosslinked, charge-modified biopolymers are incorporated into absorbent food pads. The absorbent food pads include a fluid permeable top sheet and a fluid resistant back sheet separated by an absorbent core. The top and bottom sheets include a biodegradable film, fabric, or paper, and are preferably bonded together around a periphery of the absorbent pad. Alternatively, a biodegradable adhesive, such as a starch-based or soy-based adhesive, is used to attach the absorbent core to the back sheet and top sheet. In one embodiment, the top sheet includes perforations or slits to facilitate movement of liquid from the top sheet to the absorbent core. Preferably, at least one bacterial inhibitor is included in the food pad. The at least one bacterial inhibitor is operable to be included in the top sheet, the back sheet, and/or the absorbent core. The at least one bacterial inhibitor is preferably biodegradable or biocompostable. Examples of bacterial inhibitors include organic acids, salts, biopolymers, chitosan, enzymes, and/or combinations thereof. In one embodiment, the degradation agent is included in a layer of the absorbent food pad and is operable to be mixed with the starch-based SAM upon degradation of a barrier between the absorbent core including the starch-based SAM or upon exposure to water or another activation agent. In one embodiment, the degradation agent is included in the absorbent core. Alternatively, the degradation agent is applied to the absorbent food pad separately as a liquid or a solid.

In another embodiment, the superabsorbent material is incorporated into an absorbent layer of a pet pad, with the absorbent layer of the pet pad being positioned between a fluid permeable top sheet and a fluid impermeable back sheet. The top and bottom sheets include a biodegradable film, fabric, or paper, and are preferably bonded together around a periphery of the absorbent pad. Alternatively, a biodegradable adhesive, such as a starch-based or soy-based adhesive, is used to attach the absorbent core to the back sheet and top sheet. In one embodiment, the top sheet includes perforations or slits to facilitate movement of liquid from the top sheet to the absorbent core. In one embodiment, the degradation agent is included in a layer of the absorbent food pad and is operable to be mixed with the starch-based SAM upon degradation of a barrier between the absorbent core including the starch-based SAM or upon exposure to water or another activation agent. In one embodiment, the degradation agent is included in the absorbent layer of the pet pad. Alternatively, the degradation agent is applied to the absorbent layer of the pet pad separately.

In another embodiment, the superabsorbent material is incorporated into a water barrier bag. The water barrier bag includes a liquid permeable outer shell, with the superabsorbent material being included in the shell. The liquid permeable outer shell is flexible, and the superabsorbent material is operable to swell once water or other liquid penetrates the outer shell, causing the water barrier bag to increase in size and provide a barrier against water. The degradation agent is operable to be applied as a liquid to the liquid permeable outer shell, causing the superabsorbent material to become deactivated. Alternatively, the water barrier bag is operable to be physically opened such as via cutting, and the degradation agent is applied directly to the superabsorbent material to degrade the superabsorbent material.

In yet another embodiment, the superabsorbent material is incorporated into a gel ice pack or a cold shipping pack, with the gel ice pack or the cold shipping pack including an outer liquid impermeable shell and the superabsorbent material within that shell. The gel ice pack or cold shipping pack is operable to be physically opened such as via cutting, and the degradation agent is applied directly to the superabsorbent material to degrade the superabsorbent material.

In an embodiment in which a superabsorbent material is utilized in soil amendments, the present invention provides for the degradation agent to be applied directly as a liquid to the soil including the superabsorbent material. The degradation agent is operable to degrade the superabsorbent material in the soil such that the soil does not exhibit enhanced water retention capacity compared to similar soil without superabsorbent material. Advantageously, the starch-based SAM of the present invention is operable to be treated with oxidizing agents, enzymes, bacteria, etc. which do not create environmentally toxic byproducts. Thus, the soil can be amended with superabsorbent material and treated with a degradation agent to deactivate the superabsorbent material multiple times to modify the water retention capacity of the soil as needed based on the current need for water or need to dry out the soil.

The present invention is also operable to be utilized in situations where the superabsorbent material is not incorporated into an article but is rather incorporated into a liquid or gel. In these scenarios, the degradation agent is added as a liquid or a solid to the liquid or gel including the superabsorbent material, and the superabsorbent material is deactivated such that the superabsorbent material does not swell or reswell under standard free swell conditions.

The above-mentioned examples are provided to serve the purpose of clarifying the aspects of the invention, and it will be apparent to one skilled in the art that they do not serve to limit the scope of the invention. By nature, this invention is highly adjustable, customizable and adaptable. The above-mentioned examples are just some of the many configurations that the mentioned components can take on. All modifications and improvements have been deleted herein for the sake of conciseness and readability but are properly within the scope of the present invention.

The invention claimed is:

1. A system for degrading a biodegradable superabsorbent material comprising:
an absorbent material including the biodegradable superabsorbent material; and
a degradation agent;
wherein the degradation agent is operable to permanently deactivate the particles of the biodegradable superabsorbent material such that the particles of the deactivated biodegradable superabsorbent material are not operable to swell or reswell under free swell conditions.

2. The system of claim 1, wherein at least 90% of the particles of the biodegradable superabsorbent material have a particle size of less than about 1000 microns under free swell conditions after deactivation by the degradation agent.

3. The system of claim 1, wherein the degradation agent is not operable to permanently deactivate the particles of a polyacrylate superabsorbent material.

4. The system of claim 1, wherein the absorbent material including the biodegradable superabsorbent material is flushable after exposure to the degradation agent for less than about 24 hours.

5. The system of claim 1, wherein the degradation agent includes an oxidizing agent, bacteria, and/or an enzyme.

6. The system of claim 1, where the degradation agent includes sodium hypochlorite, peroxymonosulfate, sodium percarbonate, sodium tetraborate, amylase, lipase, cellulase, and/or protease.

7. The system of claim 1, wherein the exposure to the degradation agent occurs at or below about 140° F.

8. The system of claim 1, wherein the exposure to the degradation agent occurs at about atmospheric pressure.

9. The system of claim 1, wherein the biodegradable superabsorbent material has a Free Swell Capacity (FSC) of at least 25 g/g, a Centrifuge Retention Capacity (CRC) of at least 16 g/g, and an Absorption Against Pressure (AAP) of at least 6 g/g in a saline solution before deactivation by the degradation agent.

10. A system for degrading a biodegradable superabsorbent material comprising:
an absorbent material including the biodegradable superabsorbent material and a degradation agent;
wherein the degradation agent is activated when the absorbent material is exposed to water; and
wherein the degradation agent is operable to permanently deactivate the particles of the biodegradable superabsorbent material such that the particles of the deactivated biodegradable superabsorbent material are not operable to swell or reswell under free swell conditions.

11. The system of claim 10, wherein the degradation agent is operable to permanently deactivate at least 90% of the particles of the biodegradable superabsorbent material after exposure to the degradation agent such that the at least 90% of the particles of the deactivated biodegradable superabsorbent material are not operable to swell or reswell under free swell conditions.

12. The system of claim 11, wherein the degradation agent permanently deactivates less than 10% of particles of a sodium polyacrylate superabsorbent material after exposure to the degradation agent at the same temperature and pressure and for the same time period as the deactivation of the 90% of the particles of the biodegradable superabsorbent material.

13. The system of claim 10, wherein at least 95% of the particles of the biodegradable superabsorbent material have a particle size of less than about 1000 microns under free swell conditions after deactivation by the degradation agent.

14. The system of claim 10, wherein the degradation agent includes sodium hypochlorite, peroxymonosulfate, sodium percarbonate, sodium tetraborate, amylase, lipase, cellulase, and/or protease.

15. The system of claim 10, wherein the exposure to the degradation agent occurs at less than about 140° F.

16. A system for degrading a biodegradable superabsorbent material comprising:
the biodegradable superabsorbent material; and
a degradation agent;
wherein the degradation agent is operable to permanently deactivate the particles of the biodegradable superabsorbent material such that the particles of the deactivated biodegradable superabsorbent material are not operable to swell or reswell under free swell conditions.

17. The system of claim 16, wherein at least 95% of the particles of the biodegradable superabsorbent material have a particle size of less than about 1000 microns under free swell conditions after deactivation by the degradation agent.

18. The system of claim 16, wherein the degradation agent includes an oxidizing agent, bacteria, and/or an enzyme.

19. The system of claim 16, wherein the deactivation of the biodegradable superabsorbent material occurs at or below about 140° F.

20. The system of claim 16, wherein a viscosity of a solution including the biodegradable superabsorbent material and the degradation agent decreases by at least 85% over 5 minutes.
